SEQUENCE LISTING (31 sequences)

SEQ ID NO: 1 - protein, 104 aa, Homo sapiens
MSVKMGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKK

SEQ ID NO: 2 - protein, 789 aa, Homo sapiens
MSVKMGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKKEVDATSPAPSTSSTVKTEGAEATPGAQKRKKSTKEKAGPKGSKVSEEQTQPPSPAGAGMSTAMGRSPSPKTSLSAPPNSSSTENPKTVAKCQVTPRRNVLQKRPVIVKVLSTTKPFEYETPEMEKKIMFHATVATQTQFFHVKVLNTSLKEKFNGKKIIIISDYLEYDSLLEVNEESTVSEAGPNQTFEVPNKIINRAKETLKIDILHKQASGNIVYGVFMLHKKTVNQKTTIYEIQDDRGKMDVVGTGQCHNIPCEEGDKLQLFCFRLRKKNQMSKLISEMHSFIQIKKKTNPRNNDPKSMKLPQEQRQLPYPSEASTTFPESHLRTPQMPPTTPSSSFFTKKSEDTISKMNDFMRMQILKEGSHFPGPFMTSIGPAESHPHTPQMPPSTPSSSFLTTKSEDTISKMNDFMRMQILKEGSHFPGPFMTSIGPAESHPHTPQMPPSTPSSSFLTTLKPRLKTEPEEVSIEDSAQSDLKEVMVLNATESFVYEPKEQKKMFHATVATENEVFRVKVFNIDLKEKFTPKKIIAIANYVCRNGFLEVYPFTLVADVNADRNMEIPKGLIRSASVTPKINQLCSQTKGSFVNGVFEVHKKNVRGEFTYYEIQDNTGKMEVVVHGRLTTINCEEGDKLKLTCFELAPKSGNTGELRSVIHSHIKVIKTRKNKKDILNPDSSMETSPDFFF

SEQ ID NO: 3 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 4 - protein, 20 aa, Artificial sequence
  Synthetic polypeptide
  MISC_FEATURE (1)..(20): An immunomodulatory polypeptide derived from the pyrin domain of human IFI16 with amino acid residue changes. X is selected from any natural and unnatural amino acid residues, with the proviso that X is not tyrosine (Y).
KKXKNIVLLKGLEVINDYHF

SEQ ID NO: 5 - protein, 20 aa, Artificial sequence
  Synthetic polypeptide
  MISC_FEATURE (1)..(20): An immunomodulatory polypeptide derived from the pyrin domain of human IFI16 with amino acid residue changes. X is selected from any natural and unnatural amino acid residues, with the proviso that X is not lysine (K).
KKYKNIVLLXGLEVINDYHF

SEQ ID NO: 6 - protein, 20 aa, Homo sapiens
KKYKNIVLLKGLEVINDYHF

SEQ ID NO: 7 - protein, 20 aa, Artificial sequence
  Synthetic polypeptide
  MISC_FEATURE (1)..(20): An immunomodulatory polypeptide derived from the pyrin domain of human IFI16 with amino acid residue changes.
AKYKNIVLLKGLEVINDYHF

SEQ ID NO: 8 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KAYKNIVLLKGLEVINDYHF

SEQ ID NO: 9 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKAKNIVLLKGLEVINDYHF

SEQ ID NO: 10 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYANIVLLKGLEVINDYHF

SEQ ID NO: 11 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKAIVLLKGLEVINDYHF

SEQ ID NO: 12 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNAVLLKGLEVINDYHF

SEQ ID NO: 13 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIALLKGLEVINDYHF

SEQ ID NO: 14 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVALKGLEVINDYHF

SEQ ID NO: 15 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLAKGLEVINDYHF

SEQ ID NO: 16 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLAGLEVINDYHF

SEQ ID NO: 17 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKALEVINDYHF

SEQ ID NO: 18 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKGAEVINDYHF

SEQ ID NO: 19 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKGLAVINDYHF

SEQ ID NO: 20 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKGLEAINDYHF

SEQ ID NO: 21 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKGLEVANDYHF

SEQ ID NO: 22 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKGLEVIADYHF

SEQ ID NO: 23 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKGLEVINAYHF

SEQ ID NO: 24 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKGLEVINDAHF

SEQ ID NO: 25 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKGLEVINDYAF

SEQ ID NO: 26 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKGLEVINDYHA

SEQ ID NO: 27 - protein, 20 aa, Artificial sequence
  Synthetic polypeptide
  MISC_FEATURE (1)..(20): An immunomodulatory polypeptide derived from the pyrin domain of human IFI16 with amino acid residue changes.
KKYKNIVLLKGLAVINDYAA

SEQ ID NO: 28 - protein, 31 aa, Homo sapiens
EDIPTLEDLAETLKKEKLKGRKKRRQRRRPQ

SEQ ID NO: 29 - protein, 20 aa, Artificial sequence
  Synthetic polypeptide
  MISC_FEATURE (1)..(20): An immunomodulatory polypeptide derived from the pyrin domain of human IFI16 with amino acid residue changes. X is selected from any natural and unnatural amino acid residues, with the proviso that X is not Glutamic Acid (E).
KKYKNIVLLKGLXVINDYHF

SEQ ID NO: 30 - protein, 20 aa, Artificial sequence
  Synthetic polypeptide
  MISC_FEATURE (1)..(20): An immunomodulatory polypeptide derived from the pyrin domain of human IFI16 with amino acid residue changes. X is selected from any natural and unnatural amino acid residues, with the proviso that X is not aspartic acid (D).
KKYKNIVLLKGLEVINXYHF

SEQ ID NO: 31 - protein, 20 aa, Artificial sequence
  Synthetic polypeptide
  MISC_FEATURE (1)..(20): Immunomodulatory peptide. X is selected from any natural and unnatural amino acid residues, with the proviso that X3 is not tyrosine (Y) and X10 is not lysine (K) and X13 is not glutamic acid (E) and X17 is not aspartic acid (D).
KKXKNIVLLXGLXVINXYHF

(19) United States
(12) Patent Application Publication
(10) Pub. No.: US 2020/0399335 A1
(43) Pub. Date: Dec. 24, 2020

(54) MODIFIED IMMUNOMODULATORY PEPTIDE

(71) Applicant: Aarhus Universitet, Aarhus C (DK)

(72) Inventors: Martin Roelsgaard Jakobsen, Risskov (DK); Claus Elsborg Olesen, Abyhoj (DK)

(73) Assignee: Aarhus Universitet, Aarhus C (DK)

(21) Appl. No.: 16/968,398

(22) PCT Filed: Feb. 7, 2019
(86) PCT No.: PCT/EP2019/052983

§ 371 Date: Aug. 7, 2020

(30) Foreign Application Priority Data
Feb. 8, 2018 (EP) 18155771.1
Nov. 9, 2018 (EP) 18205478.3

(51) Int. Cl. C07K 14/52; A61K 47/64; A61K 45/06; A61K 38/19; A61P 35/02; A61P 31/22

(52) U.S. Cl. CPC C07K 14/52; A61K 47/646; A61P 31/22; A61K 38/19; A61P 35/02; A61K 45/06

(57) ABSTRACT

Modified polypeptides derived from the pyrin-domain of IFI16 are provided as well as their uses in medicine. Specifically, the polypeptides are provided for use in the treatment of disorders associated with STING activity, including cancer and immunodeficient or auto-immune disorders.

### RELATED APPLICATIONS

This Application is a national stage filing under 35 U.S.C. 371 of international application number PCT/EP2019/052983, filed Feb. 7, 2019, which is incorporated herein by reference in its entirety.

### BACKGROUND

Innate immune activation by cytosolic DNA from microbial pathogens is a potent trigger of type I Interferon (IFN) and pro-inflammatory cytokines. The pathway that leads to IFN activation has been extensively studied both in terms of the proteins binding cytosolic DNA and those needed for subsequent downstream signalling and immune activation. Although multiple candidates have been suggested as sensors for cytosolic DNA, particularly two proteins have been demonstrated by separate laboratories to play a role in DNA-driven IFN responses. These are cyclic GMP-AMP synthetase (cGAS) and IFN gamma-inducible factor 16 (IFI16).

IFI16, a cytosolic and nuclear protein, has been associated with induction of type I IFN (IFN-α and IFN-β) upon stimulation with single-stranded and double-stranded DNA and by infection with different herpesviruses, human immunodeficiency virus type 1 (HIV) and bacteria. cGAS is a cytosolic protein, which is important for sensing all forms of structured DNA and recognized as the pivotal sensor of microbial DNA. It has the enzymatic capacity to produce the second messenger cyclic GMP-AMP (cGAMP), which docks onto the endoplasmic reticulum-bound protein stimulator of interferon genes (STING). This interaction induces conformational changes that allow STING to homodimerize, migrate from the ER, and recruit TANK-binding kinase 1 (TBK1). How TBK1 is actively recruited to STING is currently unknown, but absence of TBK1 binding to STING results in impaired immune activation. A recent report demonstrated that TBK1 binding to STING initiates a complex cascade of events including phosphorylation of STING as well as recruitment and activation of IFN regulatory factor 3 (IRF3). Lack of phosphorylation of STING at Ser³⁶⁶ abolishes downstream signalling and immune activation, demonstrating the importance of precise and direct activation of STING.

Studies of cGAS-deficient mice proved a clear phenotype in innate immune responses. As mice do not have a direct ortholog to human IFI16, data from IFI16-deficient mouse models are not available. Due to the lack of a definitive murine IFI16 ortholog, mouse models are poorly suitable to resolve the potential interconnection between cGAS and IFI16 in the innate immune response to foreign DNA.

In contrast to the well-described mechanism of action of cGAS in DNA sensing, there is limited knowledge on how IFI16 is related to STING-dependent signalling and also whether IFI16 may or may not be redundant to the cGAS-STING-TBK1 pathway. Previous findings have shown that the affinity of cGAS for DNA is relatively weak (Kd in the 20 uM range) and that specific sizes or structures of the DNA are required for cGAS to engage binding. Thus, it seem plausible that cGAS responds efficiently to cytosolic DNA with help from one or more co-factors.

Herpes simplex virus-1 and -2 (HSV-1 and HSV-2) are ubiquitous and highly contagious DNA viruses with the ability to establish lytic and latent infections. Innate immune sensing to HSV infection is essential for the viral controls of HSV which can lead to devastating diseases including encephalitis. HSV-1 and -2 are detected by the host protein STING in cooperation with IFI16 leading to the production of type I interferons. However, the role of IFI16 in innate immunity against HSV infection is not limited to a STING-mediated respond. IFI16 has been shown to restrict HSV-1 replication by initiating inflammasome formation, binding to HSV promotors sites, and to introduce histone modifications leading to suppression of viral DNA transcription.

Like most pathogens HSV has developed multiple mechanisms to avoid recognition by the innate immune system. This includes IFI16 degradation and inhibition of type I interferon expression.

### SUMMARY

The present invention discloses novel immunomodulatory peptides derived from the pyrin domain of human IFI16. The pyrin-domain of IFI16 is involved in the IFI16 and STING activity and the polypeptides provided herein are capable of regulating these activities and thereby modulating immunogen responses in a subject. The specific immunomodulatory activities of the polypeptides discloses herein provides an entire new approach for regulation of STING activity and thereby modulation of the innate immune response.

The immunomodulatory polypeptides derived from the pyrin domain of human IFI16 are in one aspect derived from the sequence KKYKNIVLLKGLEVINDYHF (SEQ ID NO: 6); i.e. the polypeptide may comprise the sequence KKYKNIVLLKGLEVINDYHF (SEQ ID NO: 6) or part thereof.

The polypeptide may also be a variant of SEQ ID NO: 1.

In one preferred embodiment, the polypeptide is a variant of SEQ ID NO: 6, wherein one or more of the amino acid residues at positions 2, 3, 6, 7, 8, 9, 12, 13, 15 and/or 17 remain unsubstituted. However, the amino acid residues at these positions may also be substituted with or modified to any amino acid or amino acid variant that does not alter the polarity or charge of the respective amino acid residue. Such polypeptides generally maintain the ability to induce an interferon response and are therefore suitable for inducing an immune response. Other modifications may at least partly abolish the ability of the polypeptides to induce an interferon response

In another preferred embodiment, the polypeptide is a variant of SEQ ID NO: 6, wherein one or more of the amino acid residues at positions 1, 2, 6, 7, 8, 9, 11, 12, 13, 17, 19 and/or 20 remain unsubstituted. However, the amino acid residues at these positions may also be substituted with or modified to any amino acid or amino acid variant that does not alter the polarity or charge of the respective amino acid residue. Such polypeptides generally maintain the ability to induce CXCL10 cytokine response and are therefore suitable for inducing an immune response. Other modifications may at least partly abolish the ability of the polypeptides to induce a CXCL10 cytokine response

In another preferred embodiment, the polypeptide is a variant of SEQ ID NO: 6, wherein one or more of the amino acid residues at positions 3, 5, 10, 16 and/or 17 is substituted with or modified to any amino acid or amino acid variant that alter the polarity or charge of the respective amino acid residue. In a preferred embodiment, one or more of these amino acids are substituted with alanine. Such polypeptides are generally capable of eliciting a stronger type I Interferon or cytokine response.

In another preferred embodiment, the polypeptide is a variant of SEQ ID NO: 6, wherein one or more of the amino acid residues at positions 6, 7, 14 and/or 15 remain unsubstituted. However, the amino acid residues at these positions may also be substituted with or modified to any amino acid or amino acid variant that does not alter the polarity or charge of the respective amino acid residue. Such polypeptides generally maintain the ability to induce CXCL10 cytokine response and are therefore suitable for inducing an immune response. Other modifications may at least partly abolish the antiviral effect of the polypeptide toward an HSV infection.

In another preferred embodiment, the polypeptide is a variant of SEQ ID NO: 6, wherein one or more of the amino acid residues at positions 10, 11 and/or 17 is substituted with or modified to any amino acid or amino acid variant that alter the polarity or charge of the respective amino acid residue. In a preferred embodiment, one or more of these amino acids are substituted with alanine. Such polypeptides are generally capable of eliciting a strong antiviral response against HSV infection.

In one aspect, a polypeptide peptide analog is provided, wherein the polypeptide or polypeptide analog comprises an amino acid sequence of the general formula: KKX₃KNIVLL X₁₀GLX₁₃VINX₁₇YHF (SEQ ID NO: 31), wherein X is selected from any proteinogenic (natural) and non-proteinogenic (unnatural) amino acid residues, with the proviso that X₃ is not tyrosine (Y) and X₁₀ is not lysine (K) and X₁₃ is not glutamic acid (E) and X₁₇ is not aspartic acid (D).

Preferred embodiments include polypeptides comprising or consisting of the sequence KKX₃KNIVLLKGLEVINDYHF (SEQ ID NO: 4) or KKYKNIVLLX₁₀GLEVINDYHF (SEQ ID NO: 5) KKYKNIVLLKGLX₁₃VINDYHF (SEQ ID NO: 29) or KKYKNIVLLKGLEVINX₁₇YHF (SEQ ID NO: 30) or KKYKNIVLLKGLEVINDYHF (SEQ ID NO: 6) or a fragment or homolog thereof, wherein X is selected from any proteinogenic (natural) and non-proteinogenic (unnatural) amino acid residues, with the proviso that X₃ is not tyrosine (Y) and X₁₀ is not lysine (K) and X₁₃ is not glutamic acid (E) and X₁₇ is not aspartic acid (D).

Such polypeptides are capable of modulating innate immune responses following either an IRF3 or NF-kB, or both, driven signalling caspase. In preferred embodiment, one or more of the amino acid residues at position 1, 2, 3 remain unsubstituted and/or unmodified, as modification of any of these amino acid residues can lead to increased IL6 dependent expression.

In other preferred embodiments, one or more of the amino acid residues at positions 2, 6, 7, 11, 13, 15, 16, 19 and 20 (K₂, I₆, V₇, G₁₁, E₁₃, I₁₅, N₁₆, H₁₉ and F₂₀) remain unsubstituted and/or unmodified, as modification of any of these amino acid residues can abolish partly the cytokine-dependent immunostimulatory effect of the polypeptide. However, in other preferred embodiment, one or more of the amino acid residues at positions 10, 11, 15, 16, 19 and 20 has been substituted, preferably to alanine, as such polypeptides do not elicit a cytokine-dependent immune response but strongly elicit interferon responses.

The polypeptide may also comprise one or more conjugated moieties, such as in particular a cell-penetrating peptide in either N-terminus or C-terminus of the polypeptide.

The provided polypeptides are in a particular aspect also in one aspect provided herein for use as a medicament, including for use in treatment of disorders associated with insufficient STING activity. It is also understood that the polypeptides are provided for the treatment of any disorder, which modulation of STING activity could prevent or ameliorate.

In another aspect, a method is provided of treating a disorder associated with STING activity comprising administering a polypeptide of the invention to an individual in need thereof.

### DESCRIPTION OF DRAWINGS

FIGS. 1A-1B. Screening of alanine modified peptides in THP1 cells. The monocytic cell line THP1 carries the homozygote STING mutation H₇₁A₂₃₀Q₂₉₃ suspected with decreased activity toward DNA/CDNs and found in 3% of the American population. THP1 cells were differentiated into macrophages using PMA. Two days later cells were pre-stimulated 1 hour with IFI16-STING specific polypeptide (SEQ ID NO.6) or polypeptides with single position amino acid exchange with alanine (SEQ ID NO 7 to 26). Subsequently, cells were stimulated with the STING agonist 2′3′cGAMP formulated in lipofectamine. Twenty hours later supernatants were harvest and used to assess the production of type I IFN (A) or CXCL10 (B). Data represent the mean±SD of biological triplicates, representative of two independent experiments

FIGS. 2A-2C. Screening of alanine modified peptides in primary human peripheral blood mononuclear cells (PBMCs). From two different blood donors with wildtype STING haplotype we collected PBMCs and seeded them in cultures for 4 hours. Next, cells were primed with IFI16-STING specific polypeptide (SEQ ID NO.6) or polypeptides with single position amino acid exchange with alanine (SEQ ID NO 7 to 26). Subsequently, cells were stimulated with Herring-testis DNA (HT-DNA) formulated in lipofectamine. Twenty hours later supernatants were harvest and used to assess the production of type I IFN (A-B); CXCL10 (C-D), IL-6(E-F).

FIGS. 3A-3B. Pull down of peptide with protein complexes. Cell lysates were incubated with streptavidin-beads covalently bound to IFI16-STING specific polypeptide (SEQ ID NO 6). Co-precipitation and subsequently immune blotting demonstrates that polypeptide directly binds STING, IFI16 but not TBK1 nor IRF3.

FIG. 4. Peptide stabilizes STING complex. PMA-differentiated THP1 cells were either stimulated with mock or IFI16-STING specific polypeptide (SEQ ID NO 6) and then activated with 2′3′cGAMP. Cells were lysed after 30, 60, 120, 240 and 360 minutes and used for immunoblotting as depictured in the figure.

FIG. 5. CD spectra of polypeptides with specific alanine mutations. IFI16-STING polypeptide (SEQ ID NO.6) and two specific alanine substitutions on position 11 and 13 (SEQ ID NO.17 and 19) were run through a circular dichroism spectrum to evaluate secondary structures.

FIG. 6. Triple mutated polypeptide with enhanced STING binding affinity. IFI16-STING polypeptide (SEQ ID NO. 6 and 27) were screened for IFN, CXCL10 and IL6 responses in PBMC donors stimulated with cGAMP.

FIGS. 7A-7C. IFI16-derived peptides restricts HSV-1 and HSV-2 infection in human fibroblast independently of type I interferon. FIG. 7A) Human fibroblast was infected with HSV-1 GFP (MOI 0.05 and MOI 0.1) in the presence or absence of IFI16-STING specific polypeptide (SEQ ID NO.6) or 500-1000 U/mL IFN-α. Cells were analyzed 48 hpi by flow cytometry. FIG. 7B) Human fibroblast was infected with HSV-1 GFP or HSV-2 GFP (MOI 0.05) in the presence of IFI16-STING specific polypeptide (SEQ ID NO.6). Cells were analyzed 48 hpi by flow cytometry. Data represents mean±SD of three donors run in biological duplicates. FIG. 7C) Human fibroblasts were stimulated with IFI16-STING specific polypeptide (SEQ ID NO.6) during HSV-1 GFP infection (MOI 0.1). As a positive control, human fibroblasts were stimulated with STING agonist cGAMP (5 μg/mL). Data represent the mean±SD of two donors run in biological triplicates.

FIG. 8. Cell viability test. Human fibroblast was infected with HSV-1 GFP, HSV-2 GFP (MOI 0.05) or left uninfected in the presence or absence of 80 μg/mL IFI16-STING specific polypeptide (SEQ ID NO.6). As a positive control for cell death, human fibroblast was treated with staurosporine (500 nM). Cell viability was analyzed 48 hpi. Data represent the mean±SD of three donors run in biological triplicates.

FIGS. 9A-9B. IFI16-derived peptides restricts HSV-1 and HSV-2 infection independently of STING. FIG. 9A) Human fibroblast was infected with HSV-1 GFP or HSV-2 GFP (MOI 0.05) in the presence of 40 μg/mL IFI16-STING specific polypeptide (SEQ ID NO.6). Cells were analyzed 48 hpi by flow cytometry. Data represents mean±SD of three donors run in biological duplicates. FIG. 9B) STING protein expression in utilized WT and STING KO fibroblast detected by Western blotting.

FIG. 10. IFI16-derived peptides inhibits viral VP16 expression and endogenous IFI16 degradation. Human fibroblast was infected with HSV-1 GFP or HSV-2 GFP (MOI 0.1, 1 or 5) in the presence or absence of IFI16-STING specific polypeptide (SEQ ID NO.6). 24 hpi protein expression was analyzed by western blotting using anti-VP16, anti-IFI16, anti-STING, anti-TBK1 or anti-vinculin antibodies.

FIGS. 11A-11C. Screening of alanine modified peptides in human fibroblasts. Human fibroblast was infected with HSV-1 GFP (MOI 0.05) in the presence of IFI16-STING specific polypeptide (SEQ ID NO.6) or FIG. 11A) polypeptides with single position amino acid exchange with alanine (SEQ ID NO 7 to 26). Cells were analyzed 48 hpi by flow cytometry. Data represents mean±SD of two donors run in biological duplicates. FIG. 11B) Human fibroblast was infected with HSV-1 GFP (MOI 0.1) in the presence of absence of 25 μg/mL IFI16-STING specific polypeptide (SEQ ID NO.6), polypeptide A₁₀ (SEQ ID NO.16), or Acyclovir (50 ng/mL). Cells were analyzed 48 hpi by flow cytometry. Data represents mean±SD of two donors run in biological singlets.

### DETAILED DESCRIPTION

### Definitions

The term “comprising” should be understood in an inclusive manner. Hence, by way of example, a composition comprising compound X, may comprise compound X and optionally additional compounds.

The term “polypeptide” as used herein refers to a chain of amino acid monomers linked by peptide (amide) bonds. Said chain may comprise any number of amino acid monomers, but typically comprise at least 5 amino acids. The polypeptide may comprise any amino acid, however preferably predominantly consists of naturally occurring amino acids, although one or more amino acid residues may be substituted by unnatural amino acid homologs. By naturally occurring amino acids, the following residues are meant:

| Amino acid | Three letter code | One letter code |
|---|---|---|
| alanine | ala | A |
| arginine | arg | R |
| asparagine | asn | N |
| aspartic acid | asp | D |
| asparagine or aspartic acid | asx | B |
| cysteine | cys | C |
| glutamic acid | glu | E |
| glutamine | gln | Q |
| glutamine or glutamic acid | glx | Z |
| glycine | gly | G |
| histidine | his | H |
| isoleucine | ile | I |
| leucine | leu | L |
| lysine | lys | K |
| methionine | met | M |
| phenylalanine | phe | F |
| proline | pro | P |
| serine | ser | S |
| threonine | thr | T |
| tryptophan | trp | W |
| tyrosine | tyr | Y |
| valine | val | V |

The term “polypeptide” as used herein may also refers to a chain of amino acid monomers linked by peptide (amide) bonds of non-proteinogenic origin, including but not limited to:

L-amino acids, stereoisomers of D-amino acids, β-amino acids (β³ and β²); Homo-amino acids; Proline and Pyruvic acid derivatives;

3-substituted Alanine derivatives; Glycine derivatives; Ring-substituted; phenylalanine and Tyrosine Derivatives; Linear core amino acids; N-methyl amino acids; Citrulline; Ornithine; ε-Acetyl-lysine; 3-Amino-propionic acid (β-alanine); Aminobenzoic acid; 6-Aminocaproic acid (Aca; 6-Aminohexanoic acid); Aminobutyric acid (Abu); Hydroxyproline; Mercaptopropionic acid (MPA); 3-Nitro-tyrosine; Norleucine (Nle); and Pyroglutamic acid

The term “polypeptide” as used herein may furthermore refer to a chain of amino acid monomers linked by peptide (amide) bonds of D-stereo isomers for increased stability.

Where the polypeptides disclosed herein are variants with one or more amino acid substitutions, such substitutions are in one preferred embodiment conservative mutations/substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine, a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Additionally, variants are also determined based on a predetermined number of conservative amino acid substitutions as defined herein below. Conservative amino acid substitution as used herein relates to the substitution of one amino acid (within a predetermined group of amino acids) for another amino acid (within the same group), wherein the amino acids exhibit similar or substantially similar characteristics.

Accordingly, a variant or a fragment thereof according to the invention may comprise, within the same variant of the sequence or fragments thereof, or among different variants of the sequence or fragments thereof, at least one substitution, such as a plurality of substitutions introduced independently of one another.

It is clear from the above outline that the same variant or fragment thereof may comprise more than one conservative amino acid substitution from more than one group of conservative amino acids as defined herein above.

The addition or deletion of at least one amino acid may be an addition, substitution or deletion of from preferably 2 to 15 amino acids, such as from 2 to 13 amino acids, for example from 2 to 10 amino acids, such as from 2 to 8 amino acids. Additions, substitutions or deletions of 1, 2, 3, 4, 5, 6, 7, 8, 9, 10, 11, 12, 13, 14, 15, 16, 17 or 18 amino acids are also within the scope of the present invention. Deletions and/or additions of amino acids may—independently of one another—be a deletions and/or additions within a sequence and/or at the end of a sequence.

Functional variants of polypeptides disclosed herein will be understood to exhibit amino acid sequences gradually differing from the preferred predetermined polypeptide sequence, as the number and scope of insertions, deletions and substitutions including conservative substitutions increases. This difference is measured as a reduction in sequence identity between the preferred predetermined sequence and the functional variant. All functional variants of the polypeptides disclosed herein, in particular functional variants of SEQ ID NO: 1, such as amino acids 7-26 thereof (SEQ ID NO: 6), are included within the scope of this invention, regardless of the degree of homology that they show to the respective, predetermined sequences disclosed herein, in particular SEQ IN NO: 1 or SEQ IN NO: 6. The reason for this is that some regions of the SEQ IN NO: 1 or SEQ IN NO: 6 are readily mutatable, or capable of being completely deleted, without any significant effect on the binding activity of the resulting fragment; cf. examples. E.g. amino acid positions 10 and 17 of SEQ ID NO: 6 are readily substitutable without no or substantially no effect of the function of the polypeptide in terms of efficiency against HSV treatment.

A functional variant obtained by substitution may well exhibit some form or degree of the activity of the polypeptides of SEQ IN NO: 1 or SEQ IN NO: 6, and yet be less homologous, if residues containing functionally similar amino acid side chains are substituted. Functionally similar in this respect refers to dominant characteristics of the side chains such as hydrophobic, basic, neutral or acidic, or the presence or absence of steric bulk. Accordingly, in one embodiment of the invention, the degree of identity is not a principal measure of a fragment being a variant or functional equivalent of a preferred predetermined fragment.

A non-conservative substitution leading to the formation of a functional variant would for example i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Variants obtained by substitution of amino acids may in one preferred embodiment be made based upon the hydrophobicity and hydrophilicity values and the relative similarity of the amino acid side-chain substituents, including charge, size, and the like. Exemplary amino acid substitutions which take several of the foregoing characteristics into consideration are well known to those of skill in the art and include: arginine and lysine; glutamate and aspartate; serine and threonine; glutamine and asparagine; and valine, leucine and isoleucine.

In addition to the variants described herein, sterically similar variants may be formulated to mimic the key portions of the variant structure and that such compounds may also be used in the same manner as the variants of the invention. This may be achieved by techniques of modelling and chemical designing known to those of skill in the art. It will be understood that all such sterically similar constructs fall within the scope of the present invention.

### IFI16

The polypeptides provided herein are derived from the pyrin domain of Interferon-gamma-inducible protein 16 (IFI16). IFI16 is a cytosolic and nuclear protein also known as interferon-inducible myeloid differentiation transcriptional activator. In humans IFI16 is encoded by the IFI16 gene, and the amino acid sequence of human IFI16 is provided herein as SEQ ID NO:2.

IFI16 contains several domains including a pyrin-domain, 2 HIN domains (HIN-A and HIN-B) and a BFP domain. Three isoforms of IFI16 exists, which are generated by alternative splice sites. All three isoforms contain the Pyrin and HIN domains. In one aspect, the present invention relates to polypeptides derived from the pyrin-domain of IFI16.

In human IFI16 the pyrin-domain is positioned at aa 4 to 90 of SEQ ID NO: 2. Pyrin-domains of other IFI16 proteins can be determined by aligning the IFI16 to human IFI16 of SEQ ID NO: 2 and identifying the amino acids corresponding to amino acid 4 to 90 of SEQ ID NO: 2.

The pyrin-domain of IFI16 may in particular be the pyrin-domain of human IFI16. The amino acid sequence of human IFI16 PYRIN is provided herein as SEQ ID NO: 1.

### Immunomodulatory Peptides

One aspect of the present disclosure relates to polypeptides mimicking the pyrin-domain of IFI16. The term “mimicking”, as used herein in relation to the pyrin-domain of IFI16 is meant to indicate that the relevant polypeptide is capable of exerting the same inducing effect of STING activity as full-length IFI16. Preferably, these polypeptides are capable of inducing STING activity. Thus, the polypeptides may be capable of inducing any of the STING activities described herein below in the section “IFI16 activity and STING activity”. In particular, the pyrin-domain derived polypeptides may be capable of facilitating interaction between TBK1 and STING.

The pyrin-domain derived polypeptides provided herein comprises or consists of a modified region of the pyrin-domain of IFI16 or a fragment thereof and optionally may be conjugated to a moiety. The modification of the pyrin domain region can be by way of any deletion, substitution, insertion or amino acid modification, such as a conjugation to at least one moiety, as described below.

In one aspect a polypeptide is provided, which is a variant of the pyrin-domain of IFI16 or a fragment thereof, in particular the sequence KKYKNIVLLKGLEVINDYHF (SEQ ID NO: 6) or a variant thereof, wherein one or more amino acid residues has been modified.

Variants polypeptides mentioned herein in particular include functional variants. Thus, in one preferred embodiment of the invention there is also provided variants of SEQ ID NO: 6 and variants of fragments thereof. When being polypeptides, variants are determined on the basis of their degree of identity or their homology with a predetermined amino acid sequence, said predetermined amino acid sequence being one of SEQ ID NO: 1 or SEQ ID NO: 6, or, when the variant is a fragment, a fragment of any of the aforementioned amino acid sequences, respectively.

Accordingly, variants preferably have at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined sequence.

Sequence identity is determined in one embodiment by utilising fragments of SEQ ID NO: 1 or SEQ ID NO: 6 peptides comprising at least 5 contiguous amino acids and having an amino acid sequence which is at least 80%, such as 85%, for example 90%, such as 95%, for example 99% identical to the amino acid sequence of any of SEQ ID NO: 4-30, respectively, wherein the percent identity can be determined with the algorithm GAP, BESTFIT, or FASTA in the Wisconsin Genet-ics Software Package Release 7.0, using default gap weights.

The term “sequence identity” means that two polypeptide sequences are identical (i.e., on a amino acid to amino acid basis) over the window of comparison. The term “percentage of sequence identity” is calculated by comparing two optimally aligned sequences over the window of comparison, determining the number of positions at which the identical amino acid occurs in both sequences to yield the number of matched positions, dividing the number of matched positions by the total number of positions in the window of comparison (i.e., the window size), and multiplying the result by 100 to yield the percentage of sequence identity.

The immunomodulatory polypeptides derived from the pyrin domain of human IFI16 are in one aspect derived from the sequence KKYKNIVLLKGLEVINDYHF (SEQ ID NO: 6); i.e. the polypeptide may comprise the sequence KKYKNIVLLKGLEVINDYHF (SEQ ID NO: 6) or part thereof.

The polypeptide may also be a variant of SEQ ID NO: 1.

In one preferred embodiment, the polypeptide is a variant of SEQ ID NO: 6, wherein one or more of the amino acid residues at positions 2, 3, 6, 7, 8, 9, 12, 13, 15 and/or 17 remain unsubstituted. However, the amino acid residues at these positions may also be substituted with or modified to any amino acid or amino acid variant that does not alter the polarity or charge of the respective amino acid residue. Such polypeptides generally maintain the ability to induce an interferon response and are therefore suitable for inducing an immune response. Other modifications may at least partly abolish the ability of the polypeptides to induce an interferon response

In another preferred embodiment, the polypeptide is a variant of SEQ ID NO: 6, wherein one or more of the amino acid residues at positions 1, 2, 6, 7, 8, 9, 11, 12, 13, 17, 19 and/or 20 remain unsubstituted. However, the amino acid residues at these positions may also be substituted with or modified to any amino acid or amino acid variant that does not alter the polarity or charge of the respective amino acid residue. Such polypeptides generally maintain the ability to induce CXCL10 cytokine response and are therefore suitable for inducing an immune response. Other modifications may at least partly abolish the ability of the polypeptides to induce a CXCL10 cytokine response

In another preferred embodiment, the polypeptide is a variant of SEQ ID NO: 6, wherein one or more of the amino acid residues at positions 3, 5, 10, 16 and/or 17 is substituted with or modified to any amino acid or amino acid variant that alter the polarity or charge of the respective amino acid residue. In a preferred embodiment, one or more of these amino acids are substituted with alanine. Such polypeptides are generally capable of eliciting a stronger type I Interferon or cytokine response.

In another preferred embodiment, the polypeptide is a variant of SEQ ID NO: 6, wherein one or more of the amino acid residues at positions 6, 7, 14 and/or 15 remain unsubstituted. However, the amino acid residues at these positions may also be substituted with or modified to any amino acid or amino acid variant that does not alter the polarity or charge of the respective amino acid residue. Such polypeptides generally maintain the ability to induce CXCL10 cytokine response and are therefore suitable for inducing an immune response. Other modifications may at least partly abolish the antiviral effect of the polypeptide toward an HSV infection.

In another preferred embodiment, the polypeptide is a variant of SEQ ID NO: 6, wherein one or more of the amino acid residues at positions 10, 11 and/or 17 is substituted with or modified to any amino acid or amino acid variant that alter the polarity or charge of the respective amino acid residue. In a preferred embodiment, one or more of these amino acids are substituted with alanine. Such polypeptides are generally capable of eliciting a strong antiviral response against HSV infection.

In one aspect, a polypeptide peptide analog is provided, wherein the polypeptide or polypeptide analog comprises an amino acid sequence of the general formula: KKX₃KNIVLL X₁₀GLX₁₃VINX₁₇YHF (SEQ ID NO: 31), wherein X is selected from any proteinogenic (natural) and non-proteinogenic (unnatural) amino acid residues, with the proviso that X₃ is not tyrosine (Y) and X₁₀ is not lysine (K) and X₁₃ is not glutamic acid (E) and X₁₇ is not aspartic acid (D).

In one embodiment, a polypeptide is provided, which comprises or consists of KKX₃KNIVLLKGLEVINDYHF (SEQ ID NO: 4) or a fragment thereof, wherein X is selected from any natural and unnatural amino acid residues, with the proviso that X is not tyrosine (Y), such as preferable, wherein X is amino acid residue selected from the group consisting of A, R, N, D, B, C, E, Q, Z, G, H, I, L, K, M, F, P, S, T, W and V.

In another preferred embodiment, a polypeptide is provided, which comprises or consists of KKYKNIVLLX₁₀GLEVINDYHF (SEQ ID NO: 5) or a fragment thereof, wherein X is selected from any natural and unnatural amino acid residues, with the proviso that X is not lysine (K), such as preferable, wherein X is an amino acid residue selected from the group consisting of A, R, N, D, B, C, E, Q, Z, G, H, I, L, M, F, P, S, T, W, Y and V.

The polypeptide of SEQ ID NO. 5 corresponds to SEQ ID NO: 6, where the amino acid residue at position 10 is substituted and/or modified. Modification of this amino acid residue can lead to decreased IL6 dependent expression but significantly increased type I IFN response.

In another preferred embodiment, a polypeptide is provided, which comprises or consists of KKYKNIVLLKGLX₁₃VINDYHF (SEQ ID NO: 29) or a fragment thereof, wherein X is selected from any natural and unnatural amino acid residues, with the proviso that X₁₃ is not glutamic acid (E), such as preferable, wherein X is an amino acid residue selected from the group consisting of A, R, N, D, B, C, Q, Z, G, H, I, L, K, M, F, P, S, T, W, Y and V.

In another preferred embodiment, a polypeptide is provided, which comprises or consists of KKYKNIVLLKGLEVINX₁₇YHF (SEQ ID NO: 30) or a fragment thereof, wherein X is selected from any natural and unnatural amino acid residues, with the proviso that X₁₇ is not aspartic acid (D), such as preferable, wherein X is an amino acid residue selected from the group consisting of A, R, N, E, B, C, Q, Z, G, H, I, L, K, M, F, P, S, T, W, Y and V.

In preferred embodiment, amino acid residues at position 13, 19 and 20 is substituted and/or modified (e.g. SEQ ID NO. 27, where substituted to alanine), as modification of any of these amino acid residues can lead to decreased IL6 dependent expression but significantly increased type I IFN response and CXCL10 response.

In preferred embodiment, one or more of the amino acid residues at position 1, 2, 3 remain unsubstituted and/or unmodified, as modification of any of these amino acid residues can lead to increased IL6 dependent expression.

In one embodiment, a polypeptide is provided, wherein one or more of the amino acid residues at positions 1, 2, 3, 11, 13, 15, 16, 19 and 20 (K₁, K₂, Y₃ G₁₁, E₁₃, I₁₅, N₁₆, H₁₉ and F₂₀) of SEQ ID NO: 6 remain unsubstituted and/or unmodified, whereas one or more of the remaining amino acid residues may be modified/substituted.

However, in one particular embodiment, a polypeptide is provided, wherein one or more of the amino acid residues at positions 10, 11, 13, 15, 16, 19 and 20 has been modified or substituted. The amino acid residues at positions 10, 11, 13, 15, 16, 19 and 20 can be substituted by any natural or unnatural amino acid, however in a preferred embodiment they are substituted to alanine.

In another embodiment, a variant polypeptide is provided, wherein one or more of the amino acid residues at positions 6, 7 and 14 (I₆, V₇, V₁₄, E₁₃, I₁₅, N₁₆, H₁₉ and F₂₀) of SEQ ID NO: 6 remain unsubstituted and/or unmodified, whereas one or more of the remaining amino acid residues may be modified/substituted, in particular one or more of amino acid residues at positions 1, 2, 3, 4, 10, 11, 12, 17 and/or 19.

The following polypeptides are provided as preferred embodiment, and this, the provided herein are preferably selected from the group consisting of

(SEQ ID NO: 6)
KKYKNIVLLKGLEVINDYHF (peptide 101)
(SEQ ID NO: 7)
AKYKNIVLLKGLEVINDYHF (peptide A1)
(SEQ ID NO: 8)
KAYKNIVLLKGLEVINDYHF (peptide A2)
(SEQ ID NO: 9)
KKAKNIVLLKGLEVINDYHF (peptide A3)
(SEQ ID NO: 10)
KKYANIVLLKGLEVINDYHF (peptide A4)
(SEQ ID NO: 11)
KKYKAIVLLKGLEVINDYHF (peptide A5)
(SEQ ID NO: 12)
KKYKNAVLLKGLEVINDYHF (peptide A6)
(SEQ ID NO: 13)
KKYKNIALLKGLEVINDYHF (peptide A7)
(SEQ ID NO: 14)
KKYKNIVALKGLEVINDYHF (peptide A8)
(SEQ ID NO: 15)
KKYKNIVLAKGLEVINDYHF (peptide A9)
(SEQ ID NO: 16)
KKYKNIVLLAGLEVINDYHF (peptide A10)
(SEQ ID NO: 17)
KKYKNIVLLKALEVINDYHF (peptide All)
(SEQ ID NO: 18)
KKYKNIVLLKGAEVINDYHF (peptide Al2)
(SEQ ID NO: 19)
KKYKNIVLLKGLAVINDYHF (peptide A13)
(SEQ ID NO: 20)
KKYKNIVLLKGLEAINDYHF (peptide A14)
(SEQ ID NO: 21)
KKYKNIVLLKGLEVANDYHF (peptide A15)
(SEQ ID NO: 22)
KKYKNIVLLKGLEVIADYHF (peptide A16)
(SEQ ID NO: 23)
KKYKNIVLLKGLEVINAYHF (peptide A17)
(SEQ ID NO: 24)
KKYKNIVLLKGLEVINDAHF (peptide A18)
(SEQ ID NO: 25)
KKYKNIVLLKGLEVINDYAF (peptide A19)
(SEQ ID NO: 26)
KKYKNIVLLKGLEVINDYHA (peptide A20)
(SEQ ID NO: 27)
KKYKNIVLLKGLAVINDYAA (peptide 107)
or a functional fragment of any of the above

As indicated, the polypeptides provided herein also include functional fragments. Such fragments, generally, comprise at least 5 consecutive amino acid residues, and more preferably at least 10, such as at least 11, 12, 13, 14, such as at least 15, such as at least 20 consecutive amino acid residues.

### Additional Moieties

The polypeptide may also optionally be conjugated to at least one moiety. The at least one conjugated moieties can be attached at the N-terminus or the C-terminus or even to an amino acid sidechain of the polypeptide.

In one embodiment the conjugated moiety is a peptide, a sugar, a lipid, a cell-penetrating peptide (CPP) or any other chemical group that can be covalently linked to a polypeptide. Preferred moieties are cell-penetrating peptides (CPPs), which are short peptides that facilitate cellular intake/uptake of the polypeptide. CPPs typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. CPPs can mediate cell penetration through different pathways, such as be direct penetration, endocytosis-mediated translocation, or translocation through the formation of a transitory structure (e.g. inverted micelles).

In one preferred embodiment, the CPP is the HIV TAT sequence or a modification thereof. In another embodiment, the CPP is an arginine sequence of (N₆-N₉).

The conjugated moiety may also improve physical properties of the polypeptide, such as its solubility, stability or half-life. In one embodiment, the conjugated moiety is a detectable moiety that could be used for imaging of the polypeptide; for example, the conjugated moiety is a biotin molecule. Specifically, the polypeptide may be conjugated to one or more fatty acids or fatty acid-like moieties in order to prolong in vivo half-life.

Thus, in one embodiment the conjugated moiety is modifications or any other chemical group that can support the stability of a polypeptide secondary structure of an alpha-helix.

In one embodiment, the conjugated moiety may be a compound that masks the polypeptide from the host immune system, such as a polyethylene glycol (PEG) polymer chain or a modified PEG, for example NPEG. PEG or modified PEG may also prolong the in vivo half-life of the peptide.

In yet another embodiment, the polypeptide comprises an albumin-binding domain.

In one preferred embodiment, the polypeptide comprises a N or C-terminal CPP conjugated moiety.

Peptides of the present invention may be manufactured by standard chemical synthetic methods, or by using recombinant expression systems, or by any other suitable state-of-the-art method. Thus, the peptides of the invention may be synthesized in a number of ways, including, inter alia, methods comprising:

(a) synthesizing the peptide by means of solid-phase or liquid-phase methodology, either stepwise or by fragment assembly, and isolating and purifying the final peptide product; or
(b) expressing a nucleic acid construct that encodes the peptide in a host cell, and recovering the expression product from the host cell culture; or
(c) effecting cell-free in vitro expression of a nucleic acid construct that encodes the peptide, and recovering the expression product;
or employing any combination of methods as in (a), (b) and (c) to obtain fragments of the peptide, subsequently joining (e.g., ligating) the fragments to obtain the complete peptide, and recovering the peptide.

It may be preferable to synthesize compounds of the invention by means of solid-phase or liquid-phase peptide synthesis, the methodology of which is well known to persons of ordinary skill in the art of peptide synthesis. Reference may also be made in this respect to, for example, Fields, G. B. et al., 2002, “Principles and practice of solid-phase peptide synthesis” in: Synthetic Peptides (2nd Edition), and examples provided therein.

In one embodiment, the polypeptides are synthesized on a peptide synthesizer using standard Fmoc-peptide synthesis, using HBTU as activator and N-methylmorpholine as the tertiary amine during activations. NMP (n′-methyl pyrrolidone) may be used as solvent. The coupling times may be approximately 1 h at RT. The peptides may also be side-chain deprotected in TFA:EDT:TIPS:H2O 94:2:1:3. After precipitation in diethyl ether, the peptides should be dissolved, e.g. in H2O, and purified on a C18-column in water acetonitrile gradients containing 0.1% TFA. Choice of resin is within the capabilities of those of skill in the art, however, a preferred suitable resin is resin polystyrene aminomethyl-resin, which is preferable derivatized with a Rink-amide linker. Polypeptides are preferably provided with at least 90% purity.

### Administration

The polypeptides provided herein and pharmaceutical compositions comprising such peptides may be administered to a patient in need of such treatment at various sites, for example administration at sites which bypass absorption, such as in an artery or vein or in the brain, and at sites which involve absorption, such as in the skin, under the skin, in a muscle or in the abdomen. More generally, administration of pharmaceutical compositions according to the invention may be by a variety of routes of administration, such as for example parenteral, intracranial, epidermal, dermal, intratumoral or transdermal routes. In some embodiments, other routes such as lingual, sublingual, buccal, oral, vaginal or rectal may be useful. Parenteral administration (of a pharmaceutical composition of the invention) may be performed, for example, by subcutaneous, intramuscular, intraperitoneal or intravenous injection by means of a syringe, for example a pen-like syringe. Alternatively, parenteral administration can take place by means of an infusion pump, e.g. in the form of a device or system borne by a subject or patient and advantageously comprising a reservoir containing a liquid composition of the invention and an infusion pump for delivery/administration of the composition to the subject or patient, or in the form of a corresponding miniaturized device suitable for implantation within the body of the subject or patient.

### IFI16 Activity and STING Activity

The polypeptides provided herein including fragments and variants thereof are preferably functional polypeptides, meaning that they retain one or more relevant functions. Preferably, the polypeptides have one or more IFI16 activities.

IFI16 is for example capable of interacting with the endoplasmic reticulum-bound protein stimulator of interferon genes (STING). The amino acid sequence of human STING is provided herein as SEQ ID NO: 3.

Thus, in one embodiment the functional polypeptides provided herein are capable of interacting with STING. Preferably, the polypeptides are capable of increasing STING activity.

IFI16 is involved in STING activation through direct binding of cyclic-di-nucleotides (CDNs). Thus, in one embodiment the functional polypeptides provided herein are capable of inducing STING activation, in particular capable of inducing STING activation in the presence of CDNs. However, certain polypeptides are capable of inhibiting or at least reducing STING activation e.g. following the “introduction of” or “stimulation with” CDNs or any small molecule derived of or similar to CDNs.

STING activation may be determined in a number of different ways, including: STING activation may be determined by determining STING phosphorylation. Thus, the functional polypeptides provided herein are in one embodiment capable of inducing phosphorylation of STING, e.g inducing an at least 2 fold increase in phosphorylation of STING. Alternatively, the polypeptides can be capable of inhibiting or at least reducing phosphorylation of STING. Thus, preferably the polypeptides are capable of reducing phosphorylation of STING at least 2-fold. Said phosphorylation of STING may in particular be phosphorylation of Ser³⁶⁶ of STING of SEQ ID NO: 3.

Phosphorylation of STING, and particularly phosphorylation of Ser³⁶⁶ of STING of SEQ ID NO: 3 may be determined in any useful manner, for example as described herein below in Example 3.

STING activation may also be determined as activation of expression of type I IFN or inflammatory cytokines in cells capable of expressing type I IFN or cytokines. Examples of such cells include macrophages, dendritic cells, keratinocytes, fibroblasts, monocytes, epithelia cells, B cells, or NK cells. Thus, STING activation may be determined by determining expression of type I IFN or cytokines in such cells. Thus, it may be preferred that the polypeptides provided herein are capable of inducing expression of type I IFN or cytokines in such cells, e.g. inducing an at least 2 fold increase in expression of type I IFN in such cells, e.g. in macrophages. It is also preferred that polypeptides provided herein are capable of inhibiting or at least reducing expression of type I IFN or cytokines in such cells, e.g. in macrophages. Thus, in a preferable embodiment said polypeptides provided herein are capable of reducing expression of type I IFN or of cytokines from such cells, e.g. macrophages by at least 2-fold.

In another embodiment, the polypeptides provided herein are capable of eliciting a type I interferon response.

In yet another embodiment, the polypeptides provided herein are capable of eliciting an interferon response and without eliciting a cytokine response.

Expression of type I IFN or cytokines (such as but not limited to, CXCL10 and IL6) may be determined by any useful manner, for example as described herein below in Example 1 or 2.

In one embodiment, said polypeptide may be capable of eliciting an interferon response without eliciting an IL6 cytokine response but still a CXCL10 cytokine response.

STING activation may also be determined as activation of IFNβ promoter activity. Thus, it may be preferred that the polypeptides provided herein are capable of activating IFNβ promoter activity, e.g. inducing an at least 2 fold increase in IFNβ promoter activity. It is also preferred, however, that certain polypeptides provided herein are capable of inhibiting or at least reducing activity of the IFNβ promoter. Thus, preferably the polypeptides provided herein are capable of reducing activity of the IFNβ promoter by at least 2-fold.

STING activity can also be reflected by the amount of STING in the cells, which is affected by the level of STING degradation and STING production. Thus, the activity of STING can be increased by decreasing STING degradation and/or increasing the generation of new STING. Thus, STING activity may also be determined by the relative amount of STING in the cells.

### Separation of Interferon and Cytokine Responses

Specifically surprising, it has been found the interferon response and cytokine-dependent responses can be separated using certain polypeptides provided herein. Thus, the polypeptides provided herein are capable of modulating innate immune responses in various manner and not limited to only increase or decrease responses. For example, in preferred embodiments of the polypeptide comprising or consisting of the sequence KKX₃KNIVLLKGLEVINDYHF (SEQ ID NO: 4) or KKYKNIVLLX₁₀GLEVINDYHF (SEQ ID NO: 5) or KKYKNIVLLKGLX₁₃VINDYHF (SEQ ID NO: 29) or a fragment or homolog thereof, wherein X is selected from any natural and unnatural amino acid residues, with the proviso that X₃ is not tyrosine (Y) and X₁₀ is not lysine (K) and X₁₃ is not glutamic acid (E), wherein the amino acid residues at one or more of positions 11, 13, 19 and 20 has been substituted, preferably to alanine, a cytokine-dependent immune response is abolished while eliciting a strong interferon responses.

Such IFI16 pyrin-domain polypeptides, which are capable of eliciting a strong interferon response, while repressing or at least not simultaneously eliciting a cytokine response (such as but not limited to CXCL10 and IL6), are very useful in the treatment of certain physiological conditions, including clinical conditions, such as cancer or infectious diseases where increased T cell activation or increased antiviral activity is required.

Specifically preferred are polypeptides A10 (SEQ ID NO: 16), A13 (SEQ ID NO: 19), 101 (SEQ ID NO. 6) and/or 107 (SEQ ID NO. 27), or a functional fragments or homologs thereof.

Disorder Associated with STING Activity

In one aspect, the polypeptides provided are also provided for use as a medicament, in particular for use in the treatment of a disorder associated with STING activity, more specifically disorders associated with insufficient STING activity and/or disorders, which can be treated, prevented or ameliorated by increasing STING activity.

Thus, the polypeptides provided herein are in one preferred embodiment provided for use in the treatment of a disorder associated with insufficient STING activity, which in the present context is meant to also include any disorder, which can be treated or ameliorated by increasing STING activity.

As mentioned elsewhere herein, the pyrin-domain of IFI16 can induce STING activity. Accordingly, the polypeptides provided herein are useful for treating disorders associated with insufficient STING activity, including any disorder, which can be treated or ameliorated by increasing STING activity.

In one embodiment, the disorder is associated with TBK1 and/or IRF3 and/or NF-kB activity.

In another embodiment the disorder is cancer. Cancer (malignant neoplasm) is a class of diseases in which a group of cells display the traits of uncontrolled growth (growth and division beyond the normal limits), invasion (intrusion on and destruction of adjacent tissues), and sometimes metastasis (spread to other locations in the body via lymph or blood). Most cancers form a tumor but some, like leukemia, do not.

Thus, the disorder may be cancer, for example a cancer selected from the group consisting of: colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, childhood-null acute lymphoid leukemia (ALL), thymic ALL, B-cell ALL, acute myeloid leukemia, myelomonocytoid leukemia, acute megakaryocytoid leukemia, Burkitt's lymphoma, acute myeloid leukemia, chronic myeloid leukemia, and T cell leukemia, small and large non-small cell lung carcinoma, acute granulocytic leukemia, germ cell tumors, endometrial cancer, gastric cancer, cancer of the head and neck, chronic lymphoid leukemia, hairy cell leukemia and thyroid cancer.

In a preferred embodiment, a variant polypeptide is provided for use in the treatment of a disorder associated with STING activity, such as a cancer, wherein one or more of the amino acid residues at positions 2, 6, 7, 11, 15, 16, 19 and 20 of SEQ ID NO: 6 remain unsubstituted and/or unmodified, whereas one or more of the remaining amino acid residues may be modified/substituted, in particular one or more of amino acid residues at positions 3, 10 and 13. The variant is preferably at least 70% identical to SEQ ID NO: 6, such as at least, 75%, 80%, at least 85%, 90%, 95%, such as at least 99% identical to any of SEQ ID NO: 6-30.

The disorder may also be an infection with DNA pathogens, where IFN is deleterious. Such disorders include for example HSV, HIV, Hepatitis, HPV; malaria or listeria. In one such preferred embodiment, the disorder is a herpes simplex virus (HSV) infection, such as a HSV-1 and/or HSV-2 infection.

In a preferred embodiment, a variant polypeptide is provided for use in the treatment of an HSD infection, wherein one or more of the amino acid residues at positions 6, 7 and 14 (I₆, V₇, V₁₄) of SEQ ID NO: 6 remain unsubstituted and/or unmodified, whereas one or more of the remaining amino acid residues may be modified/substituted, in particular one or more of amino acid residues at positions 1, 2, 3, 4, 10, 11, 12, 17 and/or 19, and preferably amino acid residue 10 and/or 17. The variant is preferably at least 70% identical to SEQ ID NO: 6, such as at least, 75%, 80%, at least 85%, 90%, 95%, such as at least 99% identical to any of SEQ ID NO: 6-30.

### Method of Treatment and Combination Therapy

According to one of the aspect provided herein, a method is provided of treating a disorder associated with STING activity comprising administering any one or more of the polypeptides described herein to an individual in need thereof.

However, the polypeptides described herein and as defined elsewhere herein are also provided generally for use in medicine, i.e. for use as a medicament. These polypeptides can be used for the treatment of any clinical condition, which can be treated, prevented or ameliorated by modulation of STING activity.

In one aspect, a use is provided of the provided IFI16 pyrin domain derived polypeptides for the preparation of a medicament, for example for the preparation of a medicament for the treatment of a disorder associated with STING activity, in particular insufficient STING activity. As mentioned elsewhere, the disorder may be any clinical condition, which can be treated, prevented or ameliorated by modulation of STING activity.

The uses and methods provided herein for medical use and/or for treatment of a disorder as specified herein, may also involve a combination therapy, where the polypeptides as defined herein above are combined with at least one additional active compound. The at least one additional active compound may be administered before, concomitantly or subsequent to the administration of the one or more polypeptide.

In one preferred embodiment, the polypeptide of the present disclosure is provided for use in the treatment of cancer, and in this embodiment, administration of the polypeptide is administered together with an anticancer agent.

This agent is preferably a chemotherapeutic agent. The chemotherapeutic agent is preferably administered by systemic administration, for example by intravenous injection of a solution comprising the chemotherapeutic agent or by oral administration. The chemotherapeutic agent may be selected from alkylating agents, anti-metabolites, anti-microtubule agents, topoisomerase inhibitors and cytotoxic antibiotics.

In one embodiment, the chemotherapeutic agent is an alkylating agent. An alkylating agent is used in cancer treatment as an antineoplastic agent that attaches an alkyl group to DNA. The alkyl group is attached to the guanine base of DNA, at the number 7 nitrogen atom of the purine ring. Since cancer cells, in general, proliferate faster and with less error-correction than healthy cells, cancer cells are more sensitive to DNA damage, alkylated DNA. Dialkylating agents can react with two different 7-N-guanine residues, and monoalkylating agents can react only with one 7-N of guanine.

Examples of alkylating agents are Nitrogen mustards, such as Cyclophosphamide, Mechlorethamine or mustine (HN2) (trade name Mustargen), Uramustine or uracil mustard, Melphalan, Chlorambucil, Ifosfamide and Bendamustine.

Other examples are Nitrosoureas, such as Carmustine, Lomustine and Streptozocin. In another embodiment, the alkylating agent is an Alkyl sulfonate, such as Busulfan. In another embodiment, the agent is Thiotepa or an analogue thereof.

The chemotherapeutic agent may also be a Platinum-based chemotherapeutic agent, which acts as an alkylating agent. These agents do not have an alkyl group, but nevertheless damage DNA, by permanently coordinating to DNA to interfere with DNA repair. These agents are sometimes referred to as “alkylating-like”. Such agents include Cisplatin, Carboplatin, Nedaplatin, Oxaliplatin, Satraplatin, and Triplatin tetranitrate.

In yet another embodiment, the chemotherapeutic agent is an alkylating agent selected from procarbazine, altretamine, tetrazines, such as dacarbazine, mitozolomide and temozolomide.

In one embodiment, the chemotherapeutic agent is an alkylating agent, a topoisomerase inhibitor, such as Irinotecan, which targets type 1 topoisomerase or Etoposide, which targets type 2 topoisomerase. In another embodiment, the chemotherapeutic agent is a vascular endothelial growth factor (VEGF) inhibitor, such as Bevazizumab.

In another embodiment, the chemotherapeutic agent is selected from Nitrogen mustards, such as Cyclophosphamide, Mechlorethamine or mustine (HN2) (trade name Mustargen), Uramustine or uracil mustard, Melphalan, Chlorambucil, Ifosfamide and Bendamustine. In another embodiment, the chemotherapeutic agent is selected from Nitrosoureas, such as Carmustine, Lomustine and Streptozocin. In another embodiment, the chemotherapeutic agent is selected from Alkyl sulfonates, such as Busulfan. In another embodiment, the chemotherapeutic agent is Thiotepa or an analogue thereof. In another embodiment, the chemotherapeutic agent is selected from Platinum-based chemotherapeutic agents, such as cisplatin, carboplatin, nedaplatin, oxaliplatin, satraplatin, and triplatin tetranitrate. in another embodiment, the chemotherapeutic agent is selected from procarbazine, altretamine or tetrazines, such as dacarbazine, mitozolomide and temozolomide. in another embodiment, the chemotherapeutic agent is selected from topoisomerase inhibitors such as amsacrine, etoposide, etoposide phosphate, teniposide, doxorubicin, irinotecan, topotecan, exatecan, lurtotecan. in yet another embodiment, the chemotherapeutic agent is selected from vegf inhibitors, such as bevacizumab and ranibizumab.

Notably, the at least one additional active compound provided in the uses and methods for medical use and/or for treatment of a disorder as specified herein together with a polypeptide as defined herein above may also be a non-chemotherapeutic agent. In particular, the at least one additional active compound is in one embodiment one or more checkpoint inhibitors. Checkpoint inhibitors are generally drugs that help the body recognize and attack cancer cells.

Furthermore, the at least one additional active compound provided in the uses and methods for medical use and/or for treatment of a disorder as specified herein together with a polypeptide as defined herein above may also be a non-chemotherapeutic agent. In particular, the at least one additional active compound is in one embodiment one or more T-cell costimulatory immune modulators enhancing immune activation such as, but not limited to, 4-1BB (CD137), OX40 (CD134) and CD40. Costimulatory modulates are generally drugs that help the body recognize and attack cancer cells.

Moreover, the uses and methods provided herein for medical use and/or for treatment of a disorder as specified herein, may also involve a combination therapy, where the the polypeptide as defined herein above may be combined with radiation therapy. Thus, the polypeptides as defined herein are in certain embodiments administered, with the at least one additional active compound before, during and/or after the treated individual is subjected to radiation therapy. Provision of the polypeptide as defined herein in combination with radiation therapy serves to boost the STING-dependent immune response, which is elicited by the radiation therapy and thereby maximizing the effect of the radiation therapy.

In certain embodiments, the IFI16 pyrin-domain derived polypeptide is provided for use in the treatment of disorders associated with insufficient STING activity.

### Pharmaceutical Composition

Whilst it is possible for the polypeptides of the present invention to be administered as the raw chemical, it is preferred to present them in the form of a pharmaceutical composition. Accordingly, the present invention further provides a pharmaceutical composition, which comprises an IFI16 pyrin-domain derived polypeptide of the present invention or a pharmaceutically acceptable salt thereof, and a pharmaceutically acceptable carrier therefore. Pharmaceutical compositions are also provided, which comprises a polypeptide comprising these IFI16 pyrin-domain derived polypeptides and a pharmaceutically acceptable carrier therefore.

The pharmaceutical compositions may be prepared by conventional techniques, e.g. as described in Remington: The Science and Practice of Pharmacy 2005, Lippincott, Williams & Wilkins.

The pharmaceutically acceptable carriers can be either solid or liquid. Solid form preparations include powders, tablets, pills, capsules, cachets, suppositories, and dispersible granules. A solid carrier can be one or more excipients, which may also act as diluents, flavoring agents, solubilizers, lubricants, suspending agents, binders, preservatives, wetting agents, tablet disintegrating agents, or an encapsulating material.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The polypeptides of the present invention may be formulated for parenteral administration and may be presented in unit dose form in ampoules, pre-filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Preferably, the formulation will comprise about 0.5% to 75% by weight of the active ingredient(s) with the remainder consisting of suitable pharmaceutical excipients as described herein.

Pharmaceutically acceptable salts of the IFI16 pyrin inhibitors, where they can be prepared, are also intended to be covered by this invention. These salts will be ones that are acceptable in their application to a pharmaceutical use.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The polypeptides of the invention are in general administered in an “effective amount” or an amount necessary to achieve an “effective level” in the individual patient. When the “effective level” is used as the preferred endpoint for dosing, the actual dose and schedule can vary, depending on inter-individual differences in pharmacokinetics, drug distribution, and metabolism. The “effective level” can be defined, for example, as the blood or tissue level desired in the patient that corresponds to a concentration of the compounds or polypeptides according to the invention.

The polypeptides of the invention may be administered together with one or more other active compounds, typically with one or more other active compounds useful for treatment of the particular disorder to be treated. Thus, when the disorder is cancer, the polypeptides of the invention may be administered together with one or more anti-cancer agents.

Certain embodiments of pharmaceutical compositions of the invention, which preferably are liquid pharmaceutical compositions, may comprise a compound of the invention present in a concentration from about 0.01 mg/ml to about 50 mg/ml, such as from about 1 mg/ml to about 20 mg/ml, e.g. from about 1 mg/ml to about 10 mg/ml. In some embodiments, the composition has a pH from 2.0 to 10.0. A pharmaceutical composition of the invention may further comprise a buffer system, preservative(s), isotonicity agent(s), chelating stabilizer(s) and/or surfactant(s). Particularly useful embodiments of liquid pharmaceutical compositions of the invention are aqueous compositions, i.e. compositions comprising water. Such compositions may be in the form of an aqueous solution or an aqueous suspension. Preferred embodiments of aqueous pharmaceutical compositions of the invention are aqueous solutions. In the context of the invention the term “aqueous composition” will normally refer to a composition comprising at least 50% by weight (50% w/w) of water. Likewise, the term “aqueous solution” will normally refer to a solution comprising at least 50% w/w of water, and the term “aqueous suspension” to a suspension comprising at least 50% w/w of water. In some embodiments, a pharmaceutical composition of the invention comprises an aqueous solution of a compound (or a pharmaceutically acceptable salt or solvate thereof) of the invention present at a concentration of from 0.1 mg/ml or above, together with a buffer, the composition having a pH from about 2.0 to about 10.0, such as a pH from about 6.0 to about 8.5, e.g. from about 6.5 to about 8.5, such as from about 7.0 to about 8.5, or from about 6.5 to about 8.0. In other embodiments of a pharmaceutical composition of the invention, the pH of the composition is a pH selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4, 6, 4.7, 4.8, 4.9, 15 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.8, 9.9, and 10.0. The pH of the composition may be at least 1 pH unit from (i.e., higher or lower than) the isoelectric point of the constituent polypeptide compound of the invention, such as at least 2 pH units from (i.e., higher or lower than) the isoelectric point of the compound of the invention. In further embodiments of buffer-containing pharmaceutical compositions of the invention, the buffer or buffer substance is selected from the group consisting of: acetate buffers (e.g. sodium acetate), sodium carbonate, citrates (e.g. sodium citrate), glycylglycine, histidine, glycine, lysine, arginine, phosphates (e.g. chosen among sodium dihydrogen phosphate, disodium hydrogen phosphate and trisodium phosphate), TRIS (i.e., tris(hydroxymethyl)aminomethane), HEPES (i.e., 4-(2-hydroxyethyl)-1-piperazine-ethanesulfonic acid), BICINE (i.e., N,N-bis(2-hydroxyethyl)glycine), and TRICINE (i.e., N-[tris(hydroxymethyl)methyl]glycine), as well as succinate, malate, maleate, fumarate, tartrate, and aspartate buffers, and mixtures thereof.

### Preservative

In further embodiments of pharmaceutical compositions of the invention, the composition comprises a pharmaceutically acceptable preservative. Relevant preservatives include preservatives selected from the group consisting of: phenol, o-cresol, m-cresol, p-cresol, methyl p-hydroxybenzoate, ethyl p-hydroxybenzoate, propyl p-hydroxybenzoate, butyl p-5 hydroxybenzoate, 2-phenoxyethanol, 2-phenylethanol, benzyl alcohol, ethanol, chlorobutanol, thiomerosal, bronopol, benzoic acid, imidurea, chlorhexidine, sodium dehydroacetate, chlorocresol, benzethonium chloride, chlorphenesine [i.e. 3-(p-chlorphenoxy)propane-1,2-diol] and mixtures thereof. The preservative may be present in a concentration of from 0.1 mg/ml to 30 mg/ml, such as from 0.1 mg/ml to 20 mg/mi (e.g. from 0.1 mg/ml to 5 mg/ml, or from 5 mg/ml to 10 mg/ml, or from 10 mg/ml to 20 mg/ml) in the final liquid composition. The use of a preservative in pharmaceutical compositions is well known to the skilled worker. In this connection, reference may be made to Remington: The Science and Practice of Pharmacy, 19th edition, 1995.

Isotonicity agent

In further embodiments, a pharmaceutical composition of the invention comprises an isotonicity agent (i.e., a pharmaceutically acceptable agent which is included in the composition for the purpose of rendering the composition isotonic). In some embodiments, the composition is administered to a subject by injection. Relevant isotonicity agents include agents selected from the group consisting of: salts (e.g., sodium chloride), sugars and sugar alcohols, amino acids (including glycine, arginine, lysine, isoleucine, aspartic acid, tryptophan and threonine), alditols (including glycerol, propyleneglycol (i.e. 1,2-propanediol), 1,3-propanediol and 1,3-butanediol), polyethylene glycols (including PEG400) and mixtures thereof. Suitable sugars include mono-, di- and polysaccharides, and water-soluble glucans, such as fructose, glucose, mannose, sorbose, xylose, maltose, lactose, sucrose, trehalose, dextran, pullulan, dextrin, cyclodextrin, soluble starch, hydroxyethyl starch and carboxymethylcellulose sodium salt. In some embodiments sucrose may be employed. Suitable sugar alcohols include hydroxylated C4-C8 hydrocarbons, including mannitol, sorbitol, inositol, galacititol, dulcitol, xylitol and arabitol. In some embodiments mannitol may be employed. The sugars or sugar alcohols mentioned above may be used individually or in combination. There is no fixed limit to the amount of isotonicity agent used, as long as it is soluble in the liquid formulation, establishes isotonicity and does not adversely affect the stability of the composition. The concentration of isotonicity agent (e.g. sugar or sugar alcohol) in the final liquid composition may be, e.g., from about 1 mg/ml to about 150 mg/ml, such as from 1 mg/ml to 50 mg/ml. In particular embodiments, the concentration may be from 1 mg/ml to 7 mg/ml, or from 8 mg/ml to 24 mg/ml, or from 25 mg/ml to 50 mg/ml. The use of an isotonicity agent in pharmaceutical compositions is well known to the skilled person. In this connection, reference may be made to Remington: The Science and Practice of Pharmacy, 19th edition, 1995. In further embodiments of pharmaceutical compositions of the invention, the composition comprises a chelating agent. Relevant chelating agents include salts of ethylenediaminetetraacetic acid (EDTA), citric acid or aspartic acid, and mixtures thereof. The chelating agent may suitably be present in the final liquid composition in a concentration of from 0.1 mg/ml to 5 mg/ml, such as from 0.1 mg/ml to 2 mg/ml, or from 2 mg/ml to 5 mg/ml. The use of a chelating agent in pharmaceutical compositions is well-known to the skilled worker. In this connection, reference may be made to Remington: The Science and Practice of Pharmacy, 19th edition, 1995.

### Stabilizer

In further embodiments of pharmaceutical compositions of the invention, the composition comprises a stabilizer. The use of a stabilizer in pharmaceutical compositions is well-known to the skilled worker, and in this connection reference may be made to Remington: The Science and Practice of Pharmacy, 19th edition, 1995. Particularly useful pharmaceutical compositions of the invention are stabilized liquid compositions with therapeutically active components that include a polypeptide of the invention that may otherwise possibly exhibit aggregate formation during storage in a liquid medium. In this context, “aggregate formation” refers to physical interactions between the peptide molecules that result in formation of larger assemblies that undergo some degree of visible precipitation from the solution. As used herein, “during storage in a liquid medium” refers to the storage of a liquid composition that, once prepared, is not necessarily immediately administered to a subject. Instead, following preparation, it may be packaged for storage, either in a liquid form, in a frozen state, or in a dried form for later reconstitution into a liquid form or other form suitable for administration to a subject. As used herein, “dried form” refers to an initially liquid pharmaceutical composition or formulation that has been dried by freeze-drying (i.e., lyophilization), by spray-drying or by air-drying. Aggregate formation by a peptide during storage of a liquid pharmaceutical composition thereof can adversely affect biological activity of the peptide in question, resulting in a loss of therapeutic efficacy of the pharmaceutical composition. Furthermore, aggregate formation may cause other problems, such as blockage of tubing, membranes, or pumps if such a peptide-containing pharmaceutical composition is administered using an infusion system. Thus, peptides of the invention may be beneficial in overcoming these problems. Examples of stabilizers appropriate for incorporation in pharmaceutical compositions of the invention include, but are not limited to, the following: amino acids in their free base form or salt form, e.g. amino acids carrying a charged side chain, such as arginine, lysine, aspartic acid or glutamic acid, or amino acids such as glycine or methionine (in that incorporation of methionine may additionally inhibit oxidation of methionine residues in peptides comprising at least one methionine residue susceptible to such oxidation); certain polymers (e.g., polyethylene glycols (such as PEG 3350), polyvinylalcohol (PVA), polyvinylpyrrolidone (PVP), and carboxy-/hydroxycellulose and derivatives thereof); cyclodextrins; sulfur-containing substances (such as monothioglycerol, thioglycolic acid and 2-methylthioethanol); and surfactants (such as non-ionic surfactants, including non-ionic surfactants of the Poloxamer or Polysorbate (Tween) types. The use of a surfactant in pharmaceutical compositions is well known to the skilled worker. In this connection, reference may be made to Remington: The Science and Practice of Pharmacy, 19th edition, 1995.

### Other Types of Constituents

Additional types of constituents may also be present in pharmaceutical compositions of the present invention. Non-limiting examples of classes of such constituents include wetting agents, emulsifiers, antioxidants, bulking agents, oleaginous vehicles and proteins (e.g., human serum albumin or gelatin).

Sequences

The following sequences are cited in the present disclosure
SEQ ID NO: 1-N-terminal of human IFI16 sequence +1-100 including the pyrin-domain
MSVKMGKKYKNIVLLKGLEVINDYHFRMVKSLLSNDLKLNLKMREEYDKIQIADLMEEKFRGDAGLGKLIKIFEDIPTLED
LAETLKKEKLKVKGPALSRKRKK
SEQ ID NO: 2
gamma-interferon-inducible protein 16 isoform X1 sapiens
MSVKMGKKYKNIVLLKGLEVINDYHFRMVKSLLSNDLKLNLKMREEYDKIQIADLMEEKFRGDAGLGKLIKIFEDIPTLED
LAETLKKEKLKVKGPALSRKRKKEVDATSPAPSTSSTVKTEGAEATPGAQKRKKSTKEKAGPKGSKVSEEQTQPPSPAGAG
MSTAMGRSPSPKTSLSAPPNSSSTENPKTVAKCQVTPRRNVLQKRPVIVKVLSTTKPFEYETPEMEKKIMFHATVATQTQF
FHVKVLNTSLKEKFNGKKIIIISDYLEYDSLLEVNEESTVSEAGPNQTFEVPNKIINRAKETLKIDILHKQASGNIVYGVF
MLHKKTVNQKTTIYEIQDDRGKMDVVGTGQCHNIPCEEGDKLQLFCFRLRKKNQMSKLISEMHSFIQIKKKTNPRNNDPKS
MKLPQEQRQLPYPSEASTTFPESHLRTPQMPPTTPSSSFFTKKSEDTISKMNDFMRMQILKEGSHFPGPFMTSIGPAESHP
HTPQMPPSTPSSSFLTTKSEDTISKMNDFMRMQILKEGSHFPGPFMTSIGPAESHPHTPQMPPSTPSSSFLTTLKPRLKTE
PEEVSIEDSAQSDLKEVMVLNATESFVYEPKEQKKMFHATVATENEVFRVKVFNIDLKEKFTPKKIIAIANYVCRNGFLEV
YPFTLVADVNADRNMEIPKGLIRSASVTPKINQLCSQTKGSFVNGVFEVHKKNVRGEFTYYEIQDNTGKMEVVVHGRLTTI
NCEEGDKLKLTCFELAPKSGNTGELRSVIHSHIKVIKTRKNKKDILNPDSSMETSPDFFF
SEQ ID NO: 3
STING_TMEM173 stimulator of interferon genes protein isoform 1 sapiens
MPHSSLHPSIPCPRGHGAQKAALVLLSACLVTLWGLGEPPEHTLRYLVLHLASLQLGLLLNGVCSLAEELRHIHSRYRGSY
WRTVRACLGCPLRRGALLLLSIYFYYSLPNAVGPPFTWMLALLGLSQALNILLGLKGLAPAEISAVCEKGNFNVAHGLAWS
YYIGYLRLILPELQARIRTYNQHYNNLLRGAVSQRLYILLPLDCGVPDNLSMADPNIRFLDKLPQQTGDHAGIKDRVYSNS
IYELLENGQRAGTCVLEYATPLQTLFAMSQYSQAGFSREDRLEQAKLFCRTLEDILADAPESQNNCRLIAYQEPADDSSFS
LSQEVLRHLRQEEKEEVTVGSLKTSAVPSTSTMSQEPELLISGMEKPLPLRTDFS
SEQ ID NO: 4:
KKX₃KNIVLLKGLEVINDYHF,
wherein X is selected from any natural and unnatural amino acid residues, with
the proviso that X₃ is not tyrosine (Y)
SEQ ID NO: 5:
KKYKNIVLLX₁₀GLEVINDYHF,
wherein X is selected from any natural and unnatural amino acid residues, with
the proviso that X₁₀ is not lysine (K).
SEQ ID NO: 6: KKYKNIVLLKGLEVINDYHF (peptide 101, polypeptide derived from
pyrin domain of human IF116)
SEQ ID NO: 7: AKYKNIVLLKGLEVINDYHF (peptide A1)
SEQ ID NO: 8: KAYKNIVLLKGLEVINDYHF (peptide A2)
SEQ ID NO: 9: KKAKNIVLLKGLEVINDYHF (peptide A3)
SEQ ID NO: 10: KKYANIVLLKGLEVINDYHF (peptide A4)
SEQ ID NO: 11: KKYKAIVLLKGLEVINDYHF (peptide A5)
SEQ ID NO: 12: KKYKNAVLLKGLEVINDYHF (peptide A6)
SEQ ID NO: 13: KKYKNIALLKGLEVINDYHF (peptide A7)
SEQ ID NO: 14: KKYKNIVALKGLEVINDYHF (peptide A8)
SEQ ID NO: 15: KKYKNIVLAKGLEVINDYHF (peptide A9)
SEQ ID NO: 16: KKYKNIVLLAGLEVINDYHF (peptide A10)
SEQ ID NO: 17: KKYKNIVLLKALEVINDYHF (peptide A11)
SEQ ID NO: 18: KKYKNIVLLKGAEVINDYHF (peptide A12)
SEQ ID NO: 19: KKYKNIVLLKGLAVINDYHF (peptide A13)
SEQ ID NO: 20: KKYKNIVLLKGLEAINDYHF (peptide A14)
SEQ ID NO: 21: KKYKNIVLLKGLEVANDYHF (peptide A15)
SEQ ID NO: 22: KKYKNIVLLKGLEVIADYHF (peptide A16)
SEQ ID NO: 23: KKYKNIVLLKGLEVINAYHF (peptide A17)
SEQ ID NO: 24: KKYKNIVLLKGLEVINDAHF (peptide A18)
SEQ ID NO: 25: KKYKNIVLLKGLEVINDYAF (peptide A19)
SEQ ID NO: 26: KKYKNIVLLKGLEVINDYHA (peptide A20)
SEQ ID NO: 27: KKYKNIVLLKGLAVINDYAA (peptide 107)
SEQ ID NO: 28: Biotin-EDIPTLEDLAETLKKEKLKGRKKRRQRRRPQ-NH2 (PEPTIDE
S7, Polypeptide spanning a region within the IFI16 PYRIN domain) Sapiens
SEQ ID NO: 29:
KKYKNIVLLKGLX₁₃VINDYHF,
wherein X is selected from any natural and unnatural amino acid residues, with
the proviso that X₁₃ is not Glutamic Acid (E).
SEQ ID NO: 30:
KKYKNIVLLKGLEVINX₁₇YHF,
wherein X is selected from any natural and unnatural amino acid residues, with
the proviso that X₁₇ is not aspartic acid (D).
SEQ ID NO: 31:
KKX3KNIVLLX₁₀GLX₁₃VINX₁₇YHF,
wherein X is selected from any natural and unnatural amino acid residues, with
the proviso that X₃ is not tyrosine (Y) and X₁₀ is not lysine (K) and X₁₃ is
not glutamic acid (E) and X₁₇ is not aspartic acid (D).

### EXAMPLES

### Example 1

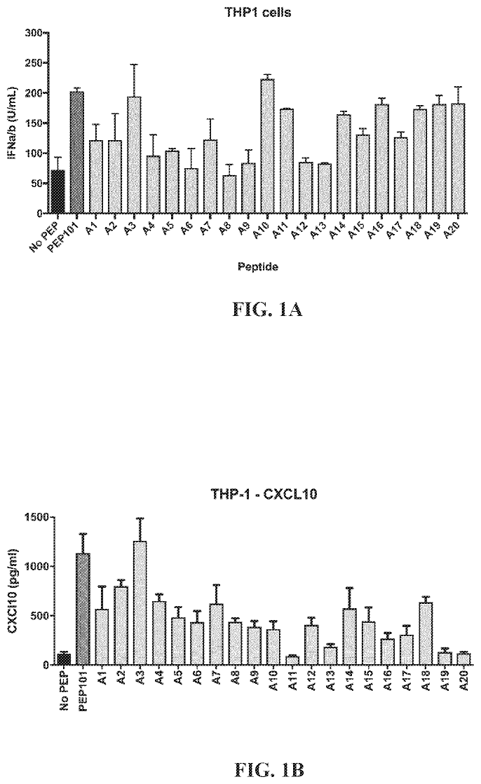

The Example shows functions of specific mutations within the polypeptide targeting the N-terminus of IFI16. Using human PMA-treated THP1 cells it was found that IFN expression, CXCL10 and IL6 expression in response to DNA and/or cGAMP was dependent on specific amino acids positioned within the polypeptide (SEQ ID NO.6). As THP1 cells carries a homozygote of STING HAQ mutant, known to be less responsive to DNA and/or cGAMP, we identified that specific alanine substitutions within the polypeptide sequence led to significant decreased IFN and/or CXCL10 responses. In particular, the amino acid positions 2, 6, 8, 9, 10, 12 and 13 affected polypeptide synergistic effects of supporting STING activation following cGAMP stimulation and the induction of type I IFNs (FIG. 1A). In contrast, the amino acid position 11, 13, 19 and 20 strongly affected the synergistic effects of supporting STING activation leading to CXCL10 cytokine secretion (FIG. 1B).

### Example 2

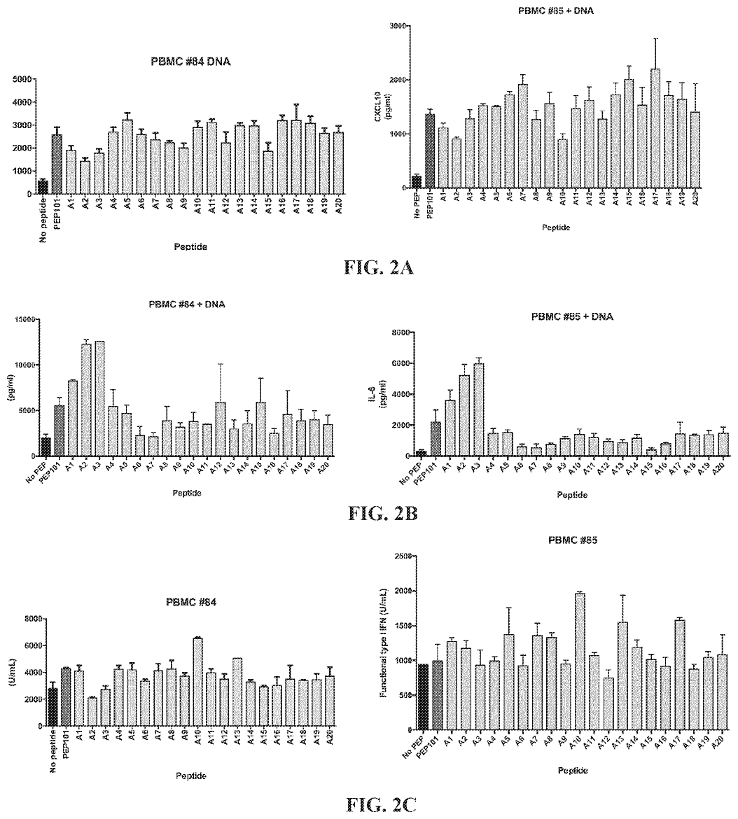

The Example shows functions of specific mutations within polypeptide targeting the N-terminus of IFI16. Using primary human PBMCs, it was found that IFN expression, CXCL10 and IL6 expression in response to DNA and/or cGAMP was dependent on specific amino acids positioned within the polypeptide (SEQ ID NO.6). Both donors carried STING wildtype proteins allowing us to evaluate important amino acid positions within the polypeptide. Surprisingly, when we evaluated the various polypeptides synergistic effects to DNA stimulation and secretion of the chemokine CXCL10, only alanine substitution on position 2 partly impaired CXCL10 in both donors (FIG. 2A). In contrast, when we evaluated the secretion of interleukin IL6, we found that alanine substitution on amino acid position 2 or 3 had a very potent and synergistic effect. However, alanine substitution on amino acid position 6, 7, 13, 15, 16 prohibited the synergistic effects of the polypeptide (FIG. 2B). More importantly, alanine substitution on amino acid position 10 and 13 significantly increased type I IFN responses and at the same time damped IL6 secretion without affecting CXL10 responses compared to the parental IFI16-STING activating polypeptide (SEQ ID NO. 5 and 29) (FIG. 2C).

### Example 3

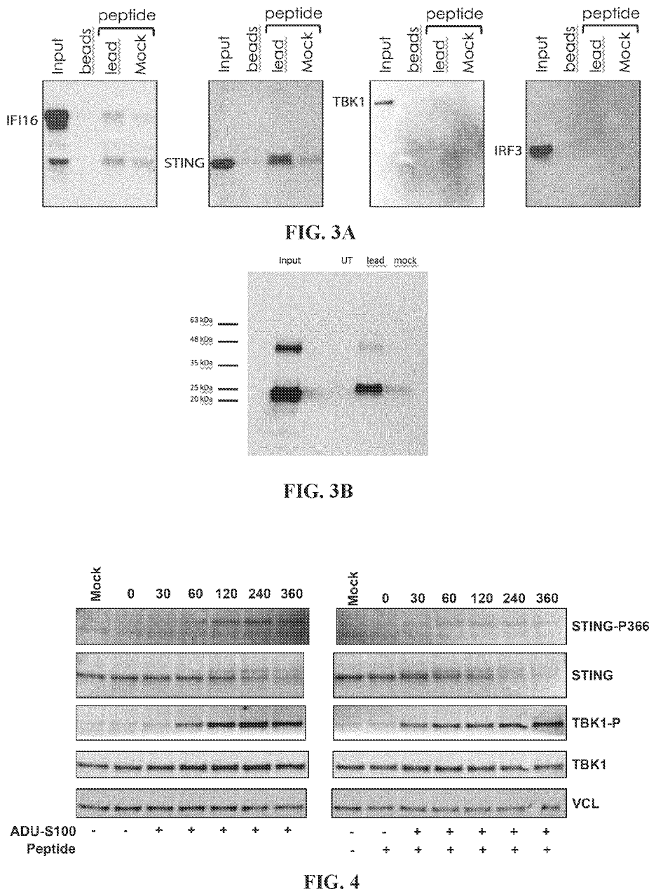

The Example shows that polypeptide (SEQ ID NO. 6) can directly interact with endogenous IFI16 and STING from THP1 cells. This was evaluated by conducting a pulldown experiment with peptide as bait and cell lysates as prey. As mock peptide, we used another polypeptide spanning a region within the IFI16 PYRIN domain (S7: Biotin-EDIPTLEDLAETLKKEKLKGRKKRRQRRRPQ-NH2; SEQ ID NO. 28) that previous has shown not to lead to synergistic effects on STING activation. Immunoblotting of eluates from the pulldown experiment demonstrate that the specific polypeptide interacted with STING as well as IFI16, but not TKB1 nor IRF3 (FIG. 3A). To confirm direct interaction with STING and not as part of a complex of proteins, we subsequently precipitated polypeptides with affinity-purified recombinant human STING protein (excluding the Transmembrane domain) and found that the polypeptide pulled-down STING even under high salt concentrations (NaCl 500 mM) (FIG. 3B).

### Example 4

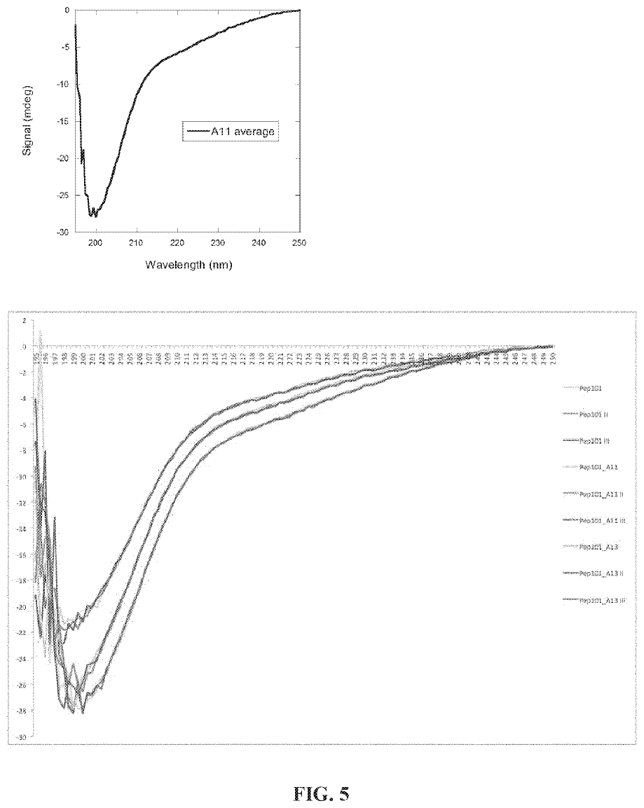

The Example shows that a possible mode-of-action by polypeptide is to stabilize STING complex, leading to a faster initiation and prolonged activation of the STING signalling cascade. We found that THP1 cells treated with cGAMP lead to robust STING activation measured by STING-5366 phosphorylation and TKB1 phosphorylation 60-120 mins post stimulation. Importantly, when cells had been pre-stimulated with polypeptide (SEQ ID NO. 6) both protein phosphorylation were significantly elevated after less than 30 mins post stimulation with cGAMP. (As shown in FIG. 4).

### Example 5

The Example illustrate the CD spectrum of polypeptide (SEQ ID NO. 6) and two alanine mutations (SEQ ID NO. 17 and 19). Based on the diagrams all three peptides depictured a secondary structure, in the recommend buffer, supporting a random coil-coil formation; cf. FIG. 5.

### Example 6

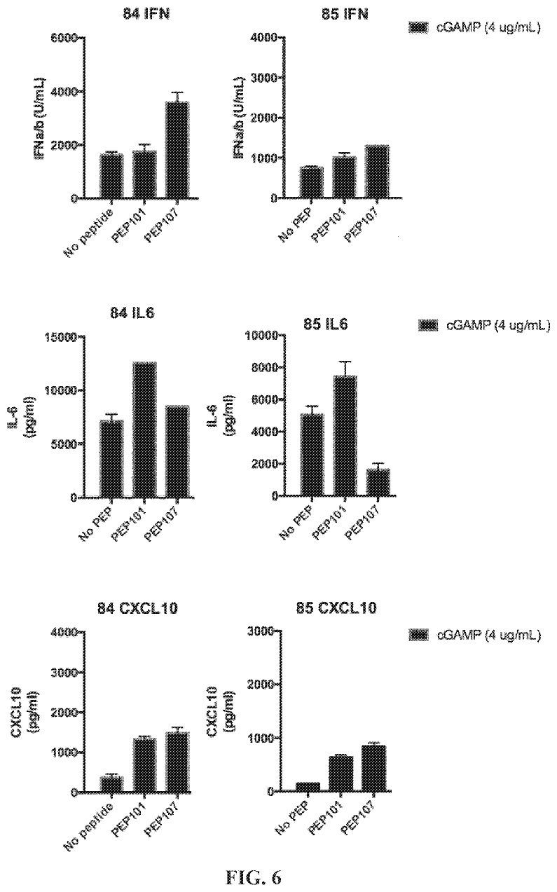

The Example show an example of the degree of synergistic STING activity by either the parental IFI16-STING activating polypeptide (peptide 101) (SEQ ID NO. 6) or a version with three alanine mutations (peptide 107) (SEQ ID NO. 27). We found that following cGAMP stimulation of PBMCs from two donors, pre-treatment with the triple mutated polypeptide impaired the IL6 response but supported increased IFN and CXCL10 secretion. (As shown in FIG. 6).

### Example 7

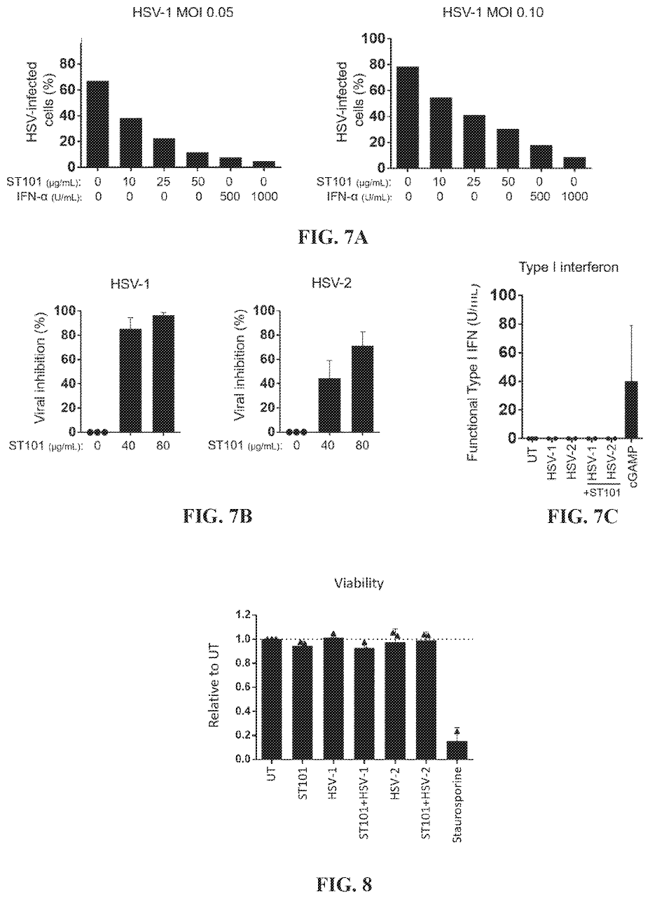

This example demonstrates that the IFI16-STING specific polypeptide (SEQ ID NO.6) can be used as a potent antiviral drug during HSV-1 and HSV-2 primary infection (FIGS. 7A-7B). The polypeptide-induced viral inhibition was found to be dose-dependent. Furthermore, data also indicates that the antiviral effect of IFI16 polypeptides was independent of the production of type I interferons (FIG. 7C).

### Example 8

The example demonstrates no toxicity during the vitro-studies of IFI16-STING specific polypeptide (SEQ ID NO.6) as an antiviral drug in human fibroblast. The highest peptide concentration utilized in the present studies reached a maximum cell death percentage of less than 10%; cf. FIG. 8.

### Example 9

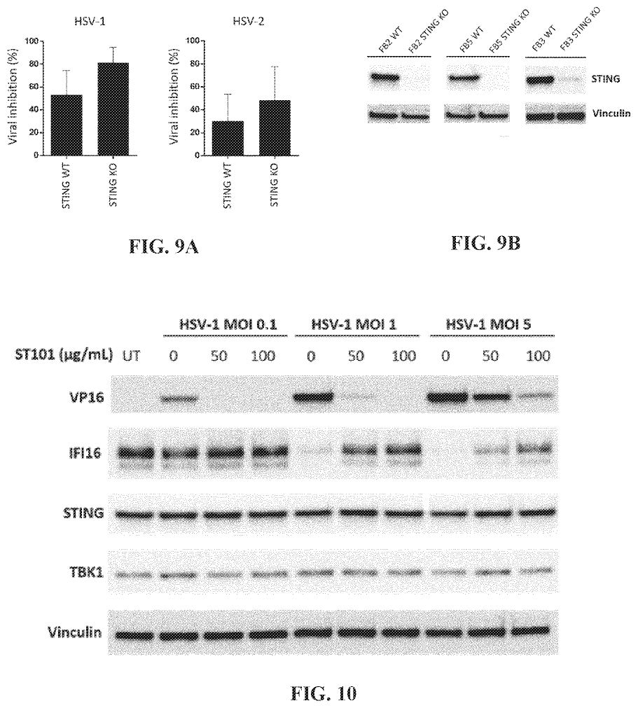

The example shows the antiviral effect of IFI16-STING specific peptide (SEQ ID NO.6) in human fibroblast depleted for STING expression. Western blot analysis confirmed nearly 100% knock-out of STING expression in the utilized donors (FIG. 9B). These data supports the finding in FIG. 1 where it is found that the antiviral effect of IFI16-STING specific polypeptide is independent of Type I interferons.

### Example 10

The example shows decreased expression of the viral protein VP16 during IFI16-STING specific polypeptide (SEQ ID NO.6) treatment in a dose-dependent manner at multiple viral MOI. This supports previous data demonstrating viral inhibition upon polypeptide treatment (FIG. 10). Further, the well-known HSV-1 induced degradation of endogenous IFI16 was clearly inhibited by the presence of IFI16-STING specific polypeptide (SEQ ID NO.6).

### Example 11

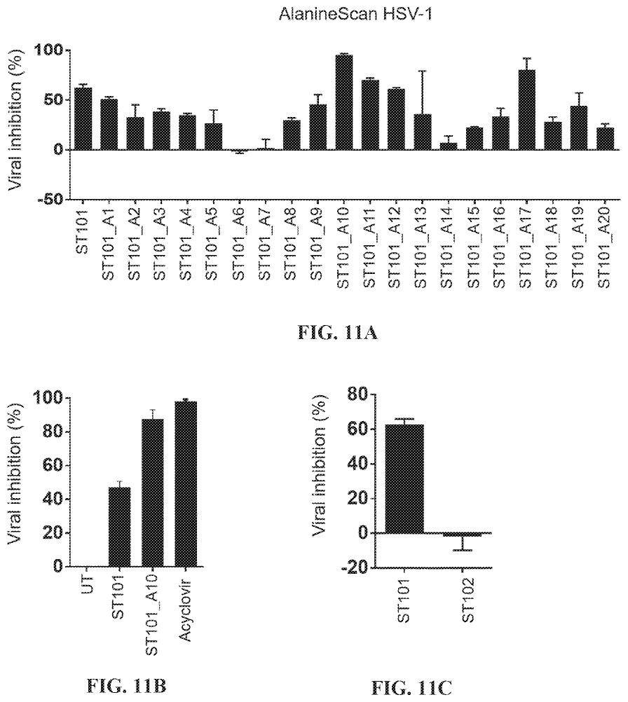

The example shows novel functions of specific mutations within the IFI16-STING specific polypeptide (SEQ ID NO.6) in human fibroblast upon HSV-1 GFP infection (FIG. 11A). The antiviral effect of the polypeptide was found to be dependent on specific amino acid position. Position 6 and 7, and 14 demonstrated depleted (6+7) or decreased (14) viral inhibition revealing these positions as significant for the antiviral effect of the polypeptide. In contrast, position 10 and 17 were found to increase the effect of the polypeptide upon amino acid substitution to nearly a 100% viral inhibition. The antiviral effect of polypeptide A10 was additional investigated using an increased MOI (0.1) (FIG. 11B). This assay confirmed an increased effect of polypeptide NO. 6 upon amino acid substitution on position 10.

### Materials and Methods

### Cell Culture.

Human acute monocytic leukemia cell line (THP-1) was cultured in RPMI 1640 (Lonza) supplemented with 10% heat inactivated fetal calf serum, 200 IU/mL Penicillin, 100 μg/mL Streptomycin and 600 μg/mL glutamine (hereafter termed RPMI complete). Mycoplasma infection was tested and ruled on a monthly basis using Lonza MycoAlert kit (LT07-703). To differentiate THP-1 cells into adherent phenotypically macrophages, cells were stimulated with 100 nM Phorbol 12-myristate 13-acetate (PMA, Sigma Aldrich 79346 5MG) in RPMI complete for 24 hours before medium was refreshed with normal RPMI complete and allowed to further differentiate an additional day (hereafter defined as macrophages).

Peripheral Blood Mononuclear cells (PBMCs) were isolated from healthy donors by Ficoll Paque gradient centrifugation (GE Healthcare). PBMCs were cultured in RPMI complete supplemented with IL-2.

Human fibroblast was cultured in DMEM 1640 (Ionza) supplemented with 10% heat inactivated fetal calf serum, 200 IU/mL Penicillin, 100 μg/mL Streptomycin and 600 μg/mL glutamine (hereafter termed DMEM complete).

### Functional Type I IFN Assay

To quantify functional type I IFN the reporter cell line HEK-Blue™ IFN-α/β (InvivoGen) was utilized according to the manufacturer's instructions. Thirty thousand HEK-Blue cells were seeded in 96-well plates with 150 μl medium devoid of Blasticidin and Zeocin and given 504 supernatant the next day. This cell line expresses secreted embryonic alkaline phosphatase under the control of the IFN-α/β inducible ISG54 promotor. SEAP activity was assessed by measuring optical density (OD) at 620 nm on a micro plate reader (ELx808, BioTEK). The standard range was made with IFN-α (A2) (PBL Assay Science).

### Enzyme-Linked Immunosorbent Assay

Protein levels of the cytokines CXCL10 and IL6 in supernatants, were measured using DuoSet ELISA kits from RnD following the manufacturer's instructions.

### Stimulation

To stimulate cells with STING agonists, 2′3′cGAMP (Invitrogene) or HT-DNA was formulated with lipofectamine2000 at a final concentration of 4 ug/ml (cGAMP) and 1 ug/ml (HT-DNA).

### Herpes Simplex Virus-Infection

Human fibroblast was infected with HSV-1 GFP (strain YK333)(7) or HSV-2 GFP (strain: 333)(8) at a multiplicity of infection (MOI) of 0.05, 0.01, 1 or 5.

Treatment with Peptides.

Each peptide was diluted in PBS pH 7 to a final concentration of 5 ug/ul. The peptides were then added to cell culture at a final concentration of 10 ug/ml. After 1 hour, cells were stimulated with STING agonists and supernatants collected after 20 hours and used for Type I IFN bioassay or cytokine ELISA. HSV-infected human fibroblast was treated with 10-80 μg/mL peptide. Peptide and HSV were added subsequently after each other.

### Pull-Down Experiments

Biotin-labelled polypeptides were immobilisered on streptavidin beads following manufactures protocol (Pierce biotinylated protein interaction pulldown kit cat no 21115). Next, beads were blocked with recombination biotin to prohibit unspecific binding. Then, prey protein capture procedure was initiated by incubated beads with either cell lysates or recombinant STING protein. After 90 minutes, beads were washed in high salt concentration ranging from 250 to 500 nM NaCL. Protein bound to peptides were finally eluted in low pH buffer (pH 2.8).

### Immunoblotting.

Whole-cell extracts or immunoprecipitation samples were analyzed by immunoblotting. Samples were diluted in XT sample buffer and XT reducing agent and ran on a SDS-PAGE (Criterion™ TGX™). Trans-Blot Turbo™ Transfer System® was used for the transfer of proteins to PVDF membranes (all reagents Bio-Rad). The membrane was blocked in 5% Difco™ skim milk (BD) or 5% bovine serum albumin (BSA) (Sigma). The antibodies used for Immunoblotting were: rabbit anti-STING (Cell Signaling, D2P2F/#13647, 1:1000), rabbit anti-pSTING (S366) (Cell SignalingTechnology, #85735), rabbit anti-TBK1 (Cell Signaling, D1 B4/#3504, 1:1000), rabbit anti-pTBK1 (Ser172) (Cell Signaling, D52C2/#5483, 1:1000), anti-IFI16 (Santa Cruz sc-6050), anti-VP16 (Abcam, ab110226, 1:1000) and anti-vinculin (Sigma Aldrich v9131). Secondary antibodies, peroxidase-conjugated F(ab′)2 donkey anti-mouse IgG (H+L), peroxidase-conjugated Affinipure F(ab′)2 donkey anti-rabbit IgG (H+L) and peroxidase conjugated F(ab′)2 donkey anti-goat IgG (H+L), were purchased from Jackson Immuno Research.

### Flow Cytometry

Human HSV-infected fibroblast was stained with LIVE/DEAD Fixable Near-IR Dead Cell Stain Kit (Thermo Fisher Scientific, L34975). Following staining, cells were fixed in 0.99% paraformaldehyde and run on a NovoCyte cytometer (ACEA Bioscience Inc.). Data was processed in FlowJo (Tree Star)

### Viability Test

Viability tests were conducted using the CellTiter-Glo® 2.0 Assay (Promega, G9242). Each sample was conducted in technical triplicates and luminescence data analyzed.

1-33. (canceled)

34. A polypeptide or polypeptide analog, wherein the polypeptide or polypeptide analog comprises an amino acid sequence of the general formula:
KKX₃KNIVLLX₁₀GLX₁₃VINX₁₇YHF (SEQ ID NO: 31)
wherein X is selected from any proteinogenic and non-proteinogenic amino acid residues, with the proviso that one or more of X₃ is not tyrosine (Y), X₁₀ is not lysine (K), X₁₃ is not glutamic acid (E) and X₁₇ is not aspartic acid (D).

35. The polypeptide or polypeptide analog according to claim 34, comprising the sequence KKX₃KNIVLLKGLEVINDYHF (SEQ ID NO: 4), KKYKNIVLLX₁₀GLEVINDYHF (SEQ ID NO: 5), KKYKNIVLLKGLX₁₃VINDYHF (SEQ ID NO: 29), or KKYKNIVLLKGLEVINX₁₇YHF (SEQ ID NO: 30), wherein X is selected from any proteinogenic and non-proteinogenic amino acid residues, with the proviso that X₃ is not tyrosine (Y) and X₁₀ is not lysine (K) and X₁₃ is not glutamic acid (E) and X₁₇ is not aspartic acid (D).

36. The polypeptide or polypeptide analog according to claim 34, wherein X is an amino acid residue selected from the group consisting of A, R, N, D, B, C, E, Q, Z, G, H, I, L, K, M, F, P, S, T, W and V.

37. The polypeptide or polypeptide analog according to claim 34, wherein one or more of the amino acid residues at one or more of the positions 2, 6, 7, 11, 15, 16, 19 and 20 (K₂, I₆, V₇, G₁₁, I₁₅, N₁₆, H₁₉ and F₂₀) remain unsubstituted and/or unmodified.

38. The polypeptide or polypeptide analog according to claim 34, wherein one or more of the amino acid residues at positions 2, 6, 7, 11, 15, 16, 19 and 20 have been substituted with alanine.

39. The polypeptide or polypeptide analog according to claim 34, wherein said polypeptide is selected from the group consisting of:
(SEQ ID NO: 9)

| | |
|---|---|
| i. | KKAKNIVLLKGLEVINDYHF (peptide A3) |

(SEQ ID NO: 16)

| | |
|---|---|
| ii. | KKYKNIVLLAGLEVINTDYHF (peptide A10) |

(SEQ ID NO: 19)

| | |
|---|---|
| iii. | KKYKNIVLLKGLAVINTDYHF (peptide A13) |

(SEQ ID NO: 23)

| | |
|---|---|
| iv. | KKYKNIVLLKGLEVINTAYHF (peptide A17) |
| and | |

(SEQ ID NO: 27)

| | | |
|---|---|---|
| v. | KKYKNIVLLKGLAVINDYAA | (peptide 107). |

40. The polypeptide or polypeptide analog according to claim 34, wherein said polypeptide is capable of eliciting a cytokine response.

41. The polypeptide or polypeptide analog according to claim 34, wherein said polypeptide is capable of eliciting a type I interferon response.

42. The polypeptide or polypeptide analog according to claim 34, wherein said polypeptide is capable of eliciting an interferon response without eliciting a cytokine response.

43. The polypeptide or polypeptide analog according to claim 34, wherein said polypeptide is capable of eliciting an interferon response without eliciting an IL6 cytokine response but still a CXCL10 cytokine response.

44. The polypeptide or polypeptide analog according to claim 43, wherein said polypeptide is peptide A10 (SEQ ID NO: 16), A13 (SEQ ID NO: 19), peptide 101 (SEQ ID NO. 6) and/or peptide 107 (SEQ ID NO. 27).

45. The polypeptide or polypeptide analog according to claim 34, wherein said polypeptide is linked to at least one conjugated moiety.

46. The polypeptide or polypeptide analog according to claim 45, wherein said at least one conjugated moiety is a cell-penetrating peptide, wherein the cell-penetrating peptide is optionally HIV TAT.

47. The polypeptide or polypeptide according to claim 34, wherein said polypeptide is capable of interacting with IFI16 and/or STING.

48. The polypeptide or polypeptide analog according to claim 34, wherein said polypeptide is capable of inducing phosphorylation of STING at Ser³⁶⁶.

49. The polypeptide or polypeptide analog according to claim 34, wherein said polypeptide is capable of increasing STING activity.

50. The polypeptide or polypeptide analog according to claim 34, wherein said polypeptide is capable of inducing STING phosphorylation.

51. A method of treating a disorder associated with STING activity or insufficient STING activity comprising administering polypeptide of claim 34 to an individual in need thereof.

52. The method of claim 51, wherein said disorder is cancer.

53. The method of claim 51, wherein said disorder is an infection with a DNA pathogen, optionally wherein the DNA pathogen is HIV, HSV-1, HSV-2, HVP, HBV, malaria or listeria.

54. The method of claim 51 further comprising administering one or more additional active compounds to the individual in need thereof.

55. The method of claim 54, wherein the additional active compound is an anti-cancer agent.

56. The method of claim 51, wherein said disorder is associated with TBK1 and/or IRF3 and/or NF-kB activity.

57. The method of claim 51, wherein said disorder is cancer, optionally wherein the cancer is induced by chronic inflammatory signalling.

58. The method of claim 57, wherein the cancer is a cutaneous skin tumour, optionally wherein the cutaneous skin tumour is basal cell carcinoma (BCC) or squamous cell carcinoma (SCC).

59. A polynucleotide encoding upon expression the polypeptide or polypeptide analog of claim 34, optionally wherein the polynucleotide is comprised within a vector.

60. A cell comprising the polynucleotide of claim 59.
